(19) United States
(12) Patent Application Publication
(10) Pub. No.: US 2023/0355127 A1
(43) Pub. Date: Nov. 9, 2023

(54) METAMATERIAL LINER FOR MRI EXCITATION

(71) Applicants: The Governors of the University of Alberta, Edmonton (CA); Alberta Health Services, Edmonton (CA)

(72) Inventors: Adam Maunder, Edmonton (CA); Ashwin Iyer, Edmonton (CA); Nicola de Zanche, Edmonton (CA)

(21) Appl. No.: 18/143,591

(22) Filed: May 4, 2023

Related U.S. Application Data
(60) Provisional application No. 63/338,446, filed on May 4, 2022.

(51) Int. Cl. A61B 5/055

(52) U.S. Cl. CPC A61B 5/055; A61B 2562/22

(57) ABSTRACT

A liner for a bore of an MRI scanner having a magnetic field. The liner comprises: plural conductors extending circumferentially within a liner region of the bore; circumferential impedances on the plural conductors; and radial impedances connecting the plural conductors to a radially outer conductive structure.

### CROSS-REFERENCE TO RELATED APPLICATIONS

This application claims priority to U.S. Provisional Patent Application No. 63/338,446, filed on May 4, 2022, which is hereby incorporated by reference in its entirety.

### TECHNICAL FIELD

Magnetic resonance imaging.

### BACKGROUND

The excitation of the magnetic resonance (MR) in MRI is typically done using localized resonators, such as volume coils, like the birdcage coil, or transmit arrays of tuned radio-frequency coils (microstrip, loop, dipoles, etc.). To produce the radio-frequency magnetic fields required for MR excitation resonators have concentrated current distributions, which produce localized tissue heating limited by safety regulations on the specific absorption rate (SAR). Travelling wave MRI uses the MRI bore (the cylindrical tube of the MRI system that the patient enters) and its conductive shield to produce a homogeneous magnetic field that is conducive to MR excitation, but with currents and corresponding electric fields that are distributed throughout more evenly, thereby reducing localized tissue heating. Traveling wave MRI requires that the radio frequency (proportional to the static magnetic field strength) is high enough, and the bore diameter is large enough, that the modes of electromagnetic propagation for MRI are above “cut-off”. The theoretical concept of a Metamaterial liner that alters the electromagnetic parameters in a thin lining surrounding the bore to enable traveling wave MRI for small diameter bores and frequencies below cut-off was developed to enable traveling wave MRI for common clinical fields strengths of 1.5 and 3 T MRI. However, further MRI liners and methods of designing/constructing liners are desired.

### SUMMARY

In an exemplary embodiment there is a liner for a bore of an MRI scanner having a magnetic field. The liner comprises: plural conductors extending circumferentially within a liner region of the bore; circumferential impedances on the plural conductors; and radial impedances connecting the plural conductors to a radially outer conductive structure.

In exemplary embodiments there may be included any one or more of the following features: the radially outer conductive structure may comprise outer conductors extending circumferentially within the bore and outer circumferential impedances on the outer conductors; the outer conductors may be connected axially by outer axial electrical connections; the outer axial electrical connections may include impedances; the radially outer conductive structure may comprise an outer conductive shield of the bore; the plural conductors may be connected axially by inner axial electrical connections; the inner axial electrical connections may include impedances; the circumferential and radial impedances may be selected to produce an effective negative and near zero permittivity within the liner region of the bore; the circumferential and radial impedances may be selected to produce a large negative permeability and effective positive and near zero permittivity within the liner region of the bore; the liner may extend less than a full length of the bore; a cable may be connected to the liner to generate an MRI excitation field within the liner; one or more additional cables may be connected to one or more respective additional points within the liner; the one or more cables may be connected to RF power supplies with different phase and/or power; the liner may be combined with an antenna within the bore to generate an MRI excitation field within the liner; the liner may be combined with plural antennas within the bore to generate an MRI excitation field within the liner, the plural antennas being connected to RF power supplies with different phase and/or power; the plural conductors may be formed in different sets, conductors of the different sets alternating within the bore, and the different sets respectively arranged to produce a desired propagating mode at respective different MR frequencies; the plural conductors may be formed in different sets, conductors of the different sets alternating within the bore, and the different sets respectively arranged to produce different propagating modes; the liner may be arranged to produce the different propagating modes at a single frequency; the different propagating modes may be separately excitable by common pulse shape with different phase and amplitude for each mode to achieve shimming of the RF magnetic field within the bore at the single frequency; the different propagating modes may also be separately excitable with different pulse shapes (parallel transmit) for example to reduce SAR or to excite a limited region of space (e.g. only the organ of interest); and the magnetic field may have a nominal strength of, for example, 1.5 T, 3 T, 4.7 T, 5 T or 7 T.

### BRIEF DESCRIPTION OF THE FIGURES

FIG. 1A is a cross-section of a metamaterial (MTM) lined cylindrical waveguide with labelled electromagnetic parameters.

FIG. 1B is a conceptual diagram of a MRI bore with a human body model and an anisotropic MTM liner.

FIG. 2 is a graph showing loci of axial permeability (μ_{z}) and radial permittivity (εᵣ) corresponding to a cutoff frequency ƒ₀=200 MHz for HEₙ₁ modes for an example liner with interior radius a=0.269 m and exterior radius b=0.280 m. Regions where solutions are too crowded together to show clearly are labelled N/A.

FIG. 3A is a diagram showing the [symbol] and [symbol] fields due to the induced current I^{ϕ} and voltage V^{sr} in a bilayer infinite current sheet.

FIG. 3B is a diagram showing the fields due to induced current and voltage in azimuthally directed rings that approximate the infinite current sheet of FIG. 3A.

FIG. 4A is a circuit representation of a single ring of a MTM liner with lumped loading (surrounded by dashed lines) and intrinsic circuit parameters.

FIG. 4B is a diagram showing a generalized periodic network model of a ladder structure corresponding to the ring of FIG. 4A, composed of N_{ϕ}ϕ—directed and r—directed lumped impedances (Z_{nϕ}^{ϕ} and Z_{nϕ}^{r}) and intrinsic series inductances (L_{nϕ}^{ϕ}) and parallel capacitances (C_{nϕ}^{r}). Excitations are represented by generators arising from the induced EMFs: V^{ϕ} from [symbol] and V^{r} from [symbol] . Subscripts indicate position and superscripts indicate orientation of connections which components are on.

FIG. 5A is a graph showing an effective μ_{z} with the mu negative and large (MNL) case for different waveguide HEₙ₁ modes.

FIG. 5B is a graph showing an effective μ_{z} with the epsilon negative and near zero (ENNZ) case for different waveguide HEₙ₁ modes.

FIG. 5C is a graph showing an effective εᵣ for the MNL case for different waveguide HEₙ₁ modes.

FIG. 5D is a graph showing an effective εᵣ for the ENNZ case for different waveguide HEₙ₁ modes. In all of FIGS. 5A-5D, the location of HEₙ₁ cutoffs are indicated by round markers on the lines.

FIG. 6A is a diagram showing an MTM liner simulation model in a transverse view with labelled lumped impedences.

FIG. 6B is a diagram showing the MTM liner simulation model of FIG. 6A in a side view with labelled lumped impedances. The single ring unit cell is outlined by the inset.

FIG. 7A is a graph showing dispersion curves (β/k₀) for the MTM lined waveguide modes for MNL cases found using the EMM (dots) or full-wave simulation (solid lines). β indicates a phase constant and k₀ indicates a free-space wavenumber.

FIG. 7B is a graph showing dispersion curves (β/k₀) for the MTM lined waveguide modes for ENNZ cases found using the EMM (dots) or full-wave simulation (solid lines). In both FIG. 7A and FIG. 7B, arrows indicate different longitudinal resonances (λ/2, λ, 3λ/2) for the HE₁₁ mode with a 80 cm long MTM liner.

FIG. 8A is a chart showing simulated transverse (subscript T) and longitudinal (subscript Z) E and H fields for MNL cases for various propagating modes for β_{Δz}=12°.

FIG. 8B is a chart showing simulated transverse (subscript T) and longitudinal (subscript Z) E and H fields for ENNZ cases for various propagating modes for β_{Δz}=12°. In both FIG. 8A and FIG. 8B, the fields are normalized to the mean of the transverse magnetic field (Hₜ) within the bore excluding the liner (r<0.25 m), designated as “μ”.

FIG. 9A is a side view of a simulation model employed in a transmission analysis.

FIG. 9B is a graph showing the transmission (red) and return loss (black) of wave-ports for a full-scale liner designed with MNL using the transmission simulation. FIG. 9C is a graph showing the transmission (red) and return loss (black) of wave-ports for a full-scale liner designed with ENNZ, obtained using the transmission analysis with the full-wave electromagnetic simulation of the structure. In FIGS. 9B and 9C the solid lines indicate transmission and reflection with only conductive losses in the copper included, while the dashed line indicates reflection and transmission with realistic losses in the inductors and capacitors (radial and azimuthial impedances) included. In other figures in this document using solid and broken lines, except where otherwise indicated, solid lines show results for a simulation model, and dashed lines show results for a network model. Horizontal arrows indicate which axis is relevant to the lines circled at the bases of the arrows.

FIG. 10A is a chart showing simulated complex magnitudes of the transverse and longitudinal H and E fields in a MTM-lined waveguide for the first four transmission peaks in the frequency-reduced MNL forward-wave propagating case.

FIG. 10B is a chart showing simulated complex magnitudes of the transverse and longitudinal H and E fields in the MTM-lined waveguide for the first four transmission peaks in the frequency-reduced ENNZ backward-wave propagating case. In both FIGS. 10A and 10B, field strength is shown in arbitrary units. Electric and magnetic field strength are shown using separate legends adjacent to the respective diagrams.

FIG. 11A is a graph showing dispersion curves (^{β}/k₀) for the MTM lined waveguide modes found using the EMM for the MNL case with variation of C^{ϕ} and L^{ϕ}.

FIG. 11B is a graph showing dispersion curves (^{β}/k₀) for the MTM lined waveguide modes found using the EMM for the MNL case with variation C^{r} and L^{r}.

FIG. 11C is a graph showing dispersion curves (^{β}/k₀) for the MTM lined waveguide modes found using the EMM for the ENNZ case with variation of C^{ϕ} and L^{ϕ}.

FIG. 11D is a graph showing dispersion curves (^{β}/k₀) for the MTM lined waveguide modes found using the EMM for the ENNZ case with variation of C^{r} and L^{r}.

FIG. 12A is a graph showing dispersion curves for the MTM lined waveguide found using the EMM with variations of the permittivity tensor: solid lines-ε⁼1=ε₀diag(εᵣ, εᵣ,1), dotted lines-ε⁼1=ε₀diag(εᵣ, 1,1), or dashed lines-ε⁼1=ε₀diag(1, εᵣ,1), applied for the MNL case.

FIG. 12B is a graph showing dispersion curves for the MTM lined waveguide found using the EMM with variations of the permittivity tensor: solid lines-ε⁼1=ε₀diag(εᵣ, εᵣ, 1), dotted lines-ε⁼1=ε₀diag(εᵣ, 1,1), or dashed lines-ε⁼1=ε₀diag(1, εᵣ,1), applied for the ENNZ case.

FIG. 13 is a rendering of an idealized MTM liner implemented in an MRI scanner bore, with human body in the center.

FIG. 14A is a diagram showing a ring of an MTM liner and a closeup of a portion of the ring.

FIG. 14B is a close-up view of a radial and longitudinal connection point to connect between adjacent rings, with labelled dimensions and geometry.

FIG. 14C is a diagram showing the ring with inset diagrams representing a simplified model with lumped Z^{z}, Z^{r} and Z^{ϕ} elements.

FIG. 15A is a diagram showing a circuit mesh model representing the network of the 2D surface of the MTM liner with the lumped circuit elements representing tuning elements incorporated in a first impedance matrix Z^{M}.

FIG. 15B is a diagram showing a circuit mesh model representing the network of the 2D surface of the MTM liner with the labelled mesh currents whose corresponding self and mutual impedance are accounted for by a second impedance matrix Z^{G}.

FIG. 16 is a diagram showing a simplified circuit model of MTM liner with L^{r}, C^{r}, C^{dr}, L^{z}, C^{z} and C^{dz} elements labelled. Dashed outlines indicate the stray capacitance components.

FIG. 17A is a graph showing dispersion diagrams of different modes derived from simulation (solid lines) or network model (dashed lines) for the ENNZ case.

FIG. 17B is a graph showing dispersion diagrams of different modes derived from simulation (solid lines) or network model (dashed lines) for the low-pass mu negative and large (LP-MNL) case.

FIG. 17C is a graph showing dispersion diagrams of different modes derived from simulation (solid lines) or network model (dashed lines) for the high-pass mu negative and large (HP-MNL) case.

FIG. 18A is a chart showing simulated E and H fields for the HP-MNL case for various propagating modes with βΔz=11.25°. The fields are normalized to the mean of the magnitude within the bore excluding the liner (r<0.24 m).

FIG. 18B is a chart showing simulated E and H fields for the LP-MNL case for various propagating modes with βΔz=11.25°. The fields are normalized to the mean of the magnitude within the bore excluding the liner (r<0.24 m).

FIG. 18C is a chart showing simulated E and H fields for the ENNZ case for various propagating modes with βΔz=11.25°. The fields are normalized to the mean of the magnitude within the bore excluding the liner (r<0.24 m).

FIG. 19A is a diagram illustrating a MTM liner used for comparison of MRI metrics with dimensions and capacitors.

FIG. 19B is a diagram illustrating a birdcage (BC) coil used for comparison of MRI metrics with dimensions and leg capacitors C_{leg} and ring capacitors C_{ring} shown.

FIG. 20A is a graph showing the reflection coefficient for a port at the end-ring of a liner with N_{z}=16 rings for the HP-MNL case simulated by network parameter analysis.

FIG. 20B is a graph showing the real and imaginary part of the input impedance for the port at the end-ring of the liner with N_{z}=16 rings for the HP-MNL case simulated by network parameter analysis. The first five longitudinal resonances (m_{z}) for the HE₁₁ mode are labelled along with the relative location of the HE₀₁, HE₁₁ and HE₂₁ mode propagation frequency ranges.

FIG. 20C is a graph showing the reflection coefficient for a port at the end-ring of a liner with N_{z}=16 rings for the HP-MNL case simulated by full wave analysis.

FIG. 20D is a graph showing the real and imaginary part of the input impedance for the port at the end-ring of the liner N_{z}=16 rings for the HP-MNL case simulated by full wave analysis. The first five longitudinal resonances (m_{z}) for the HE₁₁ mode are labelled along with the relative location of the HE₀₁, HE₁₁ and HE₂₁ mode propagation frequency ranges.

FIG. 21A is a chart showing currents (I_{ϕ}) derived from mesh network calculation on the RF ground/shield for longitudinal resonance of the HE₁₁ mode for the radial capacitance case with (L^{zM}=0).

FIG. 21B is a chart showing currents (I_{ϕ}) derived from full-wave simulation on the RF ground/shield for longitudinal resonance of the HE₁₁ mode for the radial capacitance case with (L^{zM}=0). In both FIG. 21A and FIG. 21B, currents are normalized so that a range from a maximum absolute current Iₘₐₓ and its negation −Iₘₐₓ are displayed. N_{z} indicates a sequential numbering of the ring under consideration.

FIG. 22A is a chart showing axial, coronal and saggital central slices displaying the B₁⁺ field produced inside the MRI bore when it is empty for the MTM liner design at 4.7 T-200 MHz. The fields are normalized to the mean transmit efficiency (μT/\sqrt{kW}) in the outlined torso region.

FIG. 22B is a chart showing axial, coronal and saggital central slices displaying the B₁⁺ field produced inside the MRI bore when it is empty for the BC design at 4.7 T-200 MHz. The fields are normalized to the mean transmit efficiency (μT/\sqrt{kW}) in the outlined torso region.

FIG. 23A is a chart showing central slices (respectively, axial, coronal, and sagittal) displaying the B₁⁺ field produced inside the MRI bore with human body model for the MTM liner design at 4.7 T-200 MHz. The fields are normalized to the mean transmit efficiency (μT/\sqrt{kW}).

FIG. 23B is a chart showing central slices (respectively, axial, coronal, and sagittal) displaying the B₁⁺ field produced inside the MRI bore with human body model for the BC design at 4.7 T-200 MHz. The fields are normalized to the mean transmit efficiency (μT/\sqrt{kW}).

FIG. 24A is a chart showing maximum intensity projections of localized 10 g average SAR (W/kg/μT²) with a human body model for the MTM liner design (4.7 T-200 MHz). The maximum is displayed above the axial slice.

FIG. 24B is a chart showing maximum intensity projections of localized 10 g average SAR (W/kg/μT\hat{ }2) with a human body model for the BC design (4.7 T-200 MHz). The maximum is displayed above the axial slice.

FIG. 25A is a graph showing dispersion diagrams of EH₀₁ and HEₙ₁ (HE₀₁, HE₁₁, HE₂₁, HE₃₁, HE₄₁) modes derived by full-wave simulation (dashed) or the network model (markers) as the geometric parameters are tuned to closely match the cut-offs and slope of simulation for the ENNZ case.

FIG. 25B is a graph showing dispersion diagrams of EH₀₁ and HEₙ₁ (HE₀₁, HE₁₁, HE₂₁, HE₃₁, HE₄₁) modes derived by full-wave simulation (dashed) or the network model (markers) as the geometric parameters are tuned to closely match the cut-offs and slope of simulation for the low-pass MNL case.

FIG. 25C is a graph showing dispersion diagrams of EH₀₁ and HEₙ₁ (HE₀₁, HE₁₁, HE₂₁, HE₃₁, HE₄₁) modes derived by full-wave simulation (dashed) or the network model (markers) as the geometric parameters are tuned to closely match the cut-offs and slope of simulation for the HP-MNL case.

FIG. 26A is a graph showing dispersion diagrams of different modes derived from simulation model for the ENNZ case.

FIG. 26B is a graph showing dispersion diagrams of different modes derived from network model for the ENNZ case.

FIG. 26C is a graph showing dispersion diagrams of different modes derived from simulation model for the LPMNL case.

FIG. 26D is a graph showing dispersion diagrams of different modes derived from network model for the LPMNL case.

FIG. 26E is a graph showing dispersion diagrams of different modes derived from simulation model for the HP-MNL case.

FIG. 26F is a graph showing dispersion diagrams of different modes derived from network model for the HP-MNL case. In FIGS. 26A-26F, the longitudinal impedance (L^{zM} or C^{z}) is varied to determine its influence on the dispersion slope in the three cases.

### DETAILED DESCRIPTION

Immaterial modifications may be made to the embodiments described here without departing from what is covered by the claims.

This patent document is related to U.S. Pat. No. 10,627,465 (Iyer et al.), which is hereby incorporated by reference in its entirety.

In an exemplary embodiment there is a liner 30 for a bore 34 of an MRI scanner 32 having a magnetic field. The liner comprises: plural conductors 40 extending circumferentially within a liner region of the bore; circumferential impedances 42 on the plural conductors 40; and radial impedances 38 connecting the plural conductors 40 to a radially outer conductive structure.

Embodiments of a liner for an MRI may include one or more of the following features:

The metamaterial liner 30 is constructed from circumferentially directed conductors 40 with periodic gaps connected by lumped impedances (inductors or capacitors) and joined periodically with the outer shield by impedances (inductors or capacitors)

The adjacent rings 36 can also be connected by a periodic arrangement of impedances or electrical connections, while also coupling through stray capacitance and mutual inductance.

Instead of connecting to the outer shield, the rings 36 of the metamaterial may instead connect to with another conductive structure. This outer conductor structure may follow the same structure as the rings at a larger radius, or could have different lumped elements, or no elements (just conductor or a gap in the conductor) compared to the inner structure. It could also have different dimensions (wider conductor traces or missing the axial connections for example). It could also be made of different material (e.g. aluminum vs. copper). Typically, there would still be an outer conductor shield 44 in the MRI bore 34. In many systems this outer shield is made of a non-magnetic steel mesh.

The metamaterial liner can be analyzed and theoretically described as producing a region with effective permittivity and permeability that produces the below-cutoff phenomenon for traveling wave excitation of a waveguide. The below-cutoff criteria can be achieved by choice of the tuning impedances to produce an effective near zero-permittivity that is negative or positive, a large effective permeability that is positive or negative or an effective near zero-permeability that is negative or positive in the liner region.

The required combination of effective permeability and permittivity values in the liner region required to achieve cut-off are described by analytical equations. FIG. 2 illustrates the permittivity and permeability values required for a specific waveguide and liner dimensions to achieve a cut-off frequency of 200 MHz.

The variation of the effective permittivity and permeability with frequency can be predicted, for example, based on a hybrid analytical and circuit model of the metamaterial liner that is informed by numerical simulation.

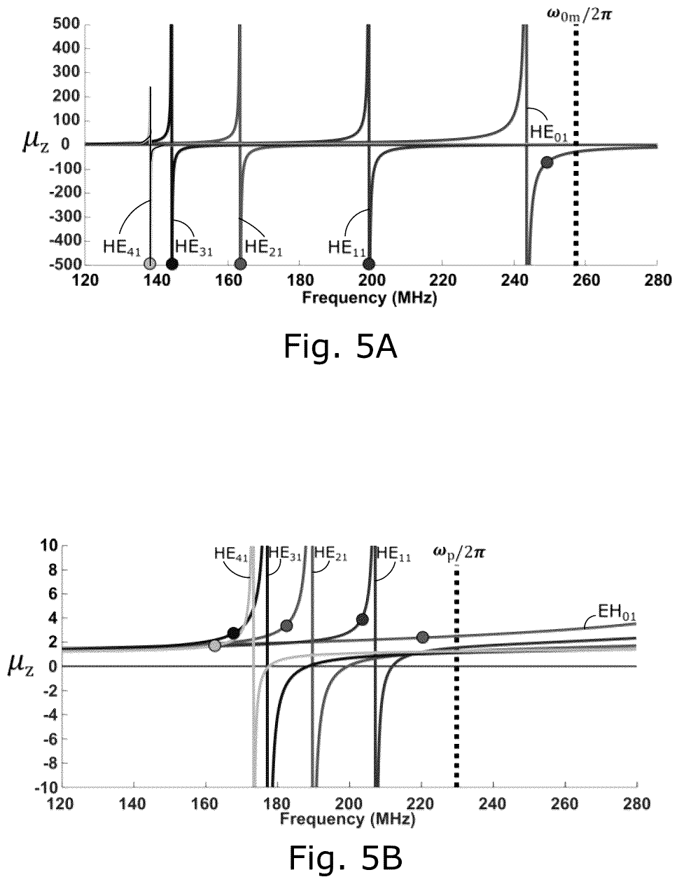

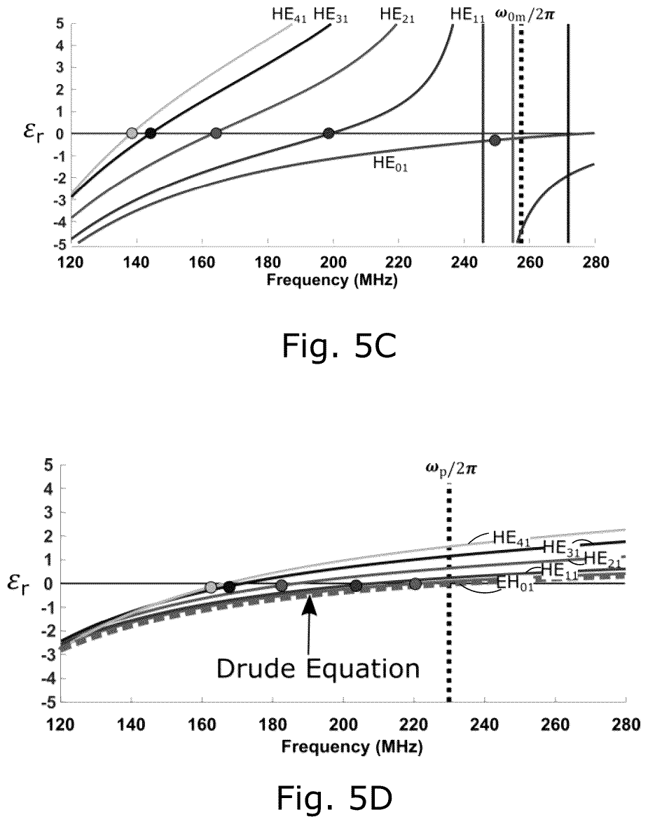

The structure of the metamaterial liner uses passive reactances for tuning and thus according to Foster's reactance theorem and the accompanying equations relating the tuning of the structure to the effective medium values this results in a permittivity and permeability that are monotonically increasing with frequency. This relation is shown in FIG. 5.

The resonant mode of the liner that is relevant for MRI excitation will occur at a frequency that is lower or higher than the cut-off frequency depending on whether the liner's propagating mode is backwards or forwards propagating, respectively.

The effective permittivity and permeability at the desired resonant mode will be different than that at the cut-off frequency. The value of the permittivity and permeability can be related to the values calculated at the frequency for which the desired dispersion or propagation constant is achieved.

In one potential implementation the cutoff occurs at the asymptote of the permeability (the MNL case in FIG. 5) where at a slightly lower frequency than cutoff the effective permeability is large and positive and permittivity is near zero and negative, while at a slightly higher frequency than cutoff, where the desired propagation is achieved, the effective permeability is large and negative and permittivity is near zero and positive.

The metamaterial liner can be tuned by choice of the tuning impedances to produce the desired dispersion and/or propagation characteristics (such as correct propagating mode) optimizing for the field distribution intensity relevant for MR excitation in the human body or test objects (phantoms).

The dispersion and propagation characteristics can be predicted, for example, based on a hybrid analytical and circuit model of the metamaterial liner that is informed by numerical simulation.

The dispersion and propagation characteristics can be predicted, for example, by a circuit/network model and method of analysis based on the induced currents.

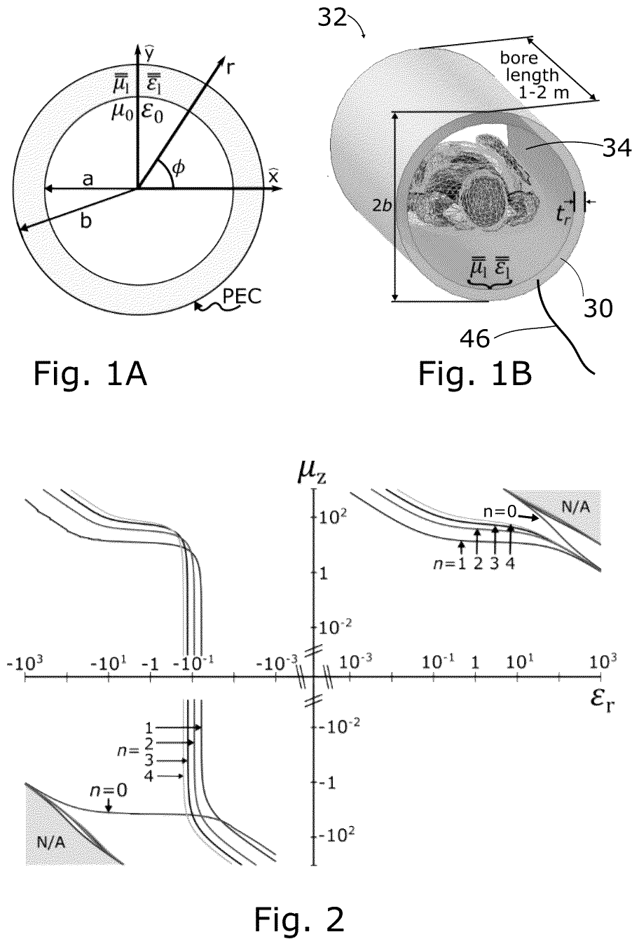

A cross section of a metamaterial (MTM) lined cylindrical waveguide with labelled electromagnetic parameters is shown in FIG. 1A.

A conceptual diagram of an MRI bore 34 with a human body model and an anisotropic MTM liner is shown in FIG. 1B.

The radial and axial extent of the metamaterial liner may be tailored to the application (e.g., to match the target anatomy or field of view determined by static B₀ and gradient fields). For example, it may be full body or head coil sized. The axial extent can be optimized for a central homogeneous field. Radial extent may also be adjusted. For example, a head coil would have a radius and axial size that closely form fits to the head, for example typically 14-17 cm vs. a whole body coil that would accommodate a plurality of all body sizes, for example typically 28-34 cm in radius. For any given metamaterial design, the frequency of propagation depends on the radius of the waveguide, so the metamaterial may be designed to enable propagation at a desired frequency while having a suitable radius.

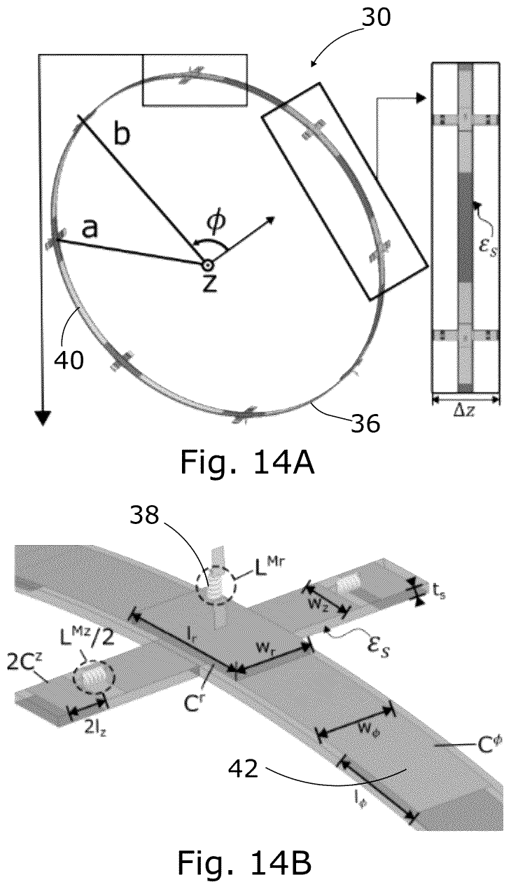

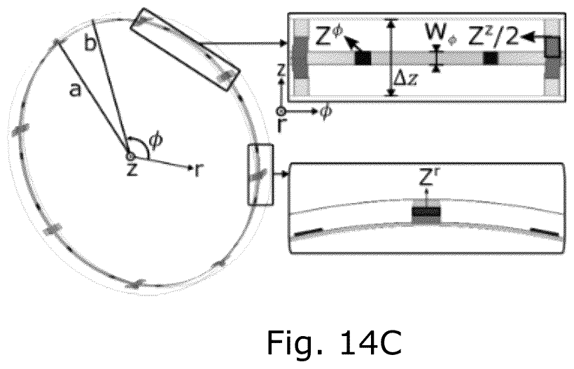

A diagram showing a ring 36 of an MTM liner 30 and a closeup of a portion of the ring is shown in FIG. 14A.

Rings with alternating tuning can be used to produce the desired propagating mode at multiple MR frequencies. Suitable frequencies may include, for example, resonant frequencies for particular types of nuclei at MRI magnetic field strengths, which are well known in the art.

Alternating rings can be used to produce different propagation modes simultaneously, such as those with different circumferential mode orders or different axial resonances, which can be excited separately for beam forming (shimming) of the radio-frequency magnetic field.

There may be 2 or more sets of rings that alternate to achieve 2 or more corresponding frequencies or propagating modes as described above.

The metamaterial liner 30 can be directly fed from a cable 46 and RF power distribution network. The cable/RF distribution network, for example, can connect in a break in the conductor 40 on the inner or outer conductive traces of the metamaterial liner rings, or it can connect in parallel or across a lumped tuning element that is one of the radially directed, axially directed or circumferentially directed lumped elements. The cable or power distribution network can for example have a ground and a hot (active) conductor, or opposite phase hot conductors. There may be plural such cables. The other end of the cable(s) connects to the RF power amplifier(s) for example via an impedance matching network or power distribution network.

The RF distribution network can comprise matching networks, power splitters, variable tuning networks and any other active or passive RF network.

The metamaterial liner may be fed from multiple points with different phase and/or power (i.e., shimming, and/or quadrature or four-point driving) to optimize the homogeneity and efficiency of the MR excitation field.

The metamaterial liner may be excited from a separate resonant structure (e.g., an antenna) coupled to it and fed radiofrequency power separately.

A head coil version has been made and tested, a second version of the prototype was constructed and preliminary testing was performed at 4.7 T.

A whole-body coil has been made and was tested in a 3 T system.

A body coil may be integrated with the bore, for example in clinical scanners (1.5 T or 3 T) or research scanners at higher field strengths (4.7 T and 7 T). Insertable versions and smaller scaled versions for the head or localized body parts (e.g. the legs) can also be made, for any field strength, including any of these values. Any other field strength may also be used. The values of 1.5, 3, 4.7 and 7 may be nominal values. For example, Siemens uses slightly lower fields (low enough, e.g., for RF coils from other manufacturers to be incompatible) even though they market their scanners at the nominal values (1.5, 3, 7, etc.).

There is disclosed further details of an exemplary liner structure, consisting of closely-spaced rings of conductor with associated distributed impedances and added lumped impedances. The liner may be represented as a material with effective permittivity and permeability, i.e. a metamaterial. An exemplary model is developed that is a hybrid of circuit theory and analytical methods that are informed by numerical simulation. The model is then tested by variation of parameters and comparing to simulation of a mock MRI bore lined with the metamaterial to facilitate traveling wave MRI at below cutoff.

The circuit model may be used to obtain accurate results for the effective medium properties of a complex periodic electromagnetic structure.

Both the method of analysis and the circuit model presented for the effective medium of the liner ring structure are believed to be novel contributions to the metamaterial field.

A behavior of the permeability is disclosed and its dependence on the circular waveguide mode order, which is a whole number describing the periodic variation of the field and current excitation phase and amplitude in the circumferential direction. The exemplary method presented employs the effective medium model and analytical metamaterial liner model to tune the structure for the correct mode excitation and dispersion to permit a travelling wave type excitation of the magnetic resonance signal for any bore size or field strength (demonstrated for 4.7 T-200 MHz and 56 cm bore diameter).

There is disclosed details of the practical and theoretical concepts underlying the design of a metamaterial (MTM) liner for magnetic resonance imaging (MRI) radio frequency (RF) excitation. There is also disclosed details of the practical design and a performance comparison to a conventional birdcage coil. The operation of the MTM liner 30 relies on specifically engineered effective permittivity and permeability of a thin liner on the inner surface of the MRI bore 34, to enable the propagation of electromagnetic (EM) waves at frequencies below the natural-cutoff of the bore itself. Here, the effective anisotropic EM properties of the MTM liner 30 structure are calculated by a combined circuit- and effective-medium model, and the analytical dispersion characteristics are compared to full-wave eigenmode simulations. The results of the effective medium model representation of the MTM liner agree fundamentally with simulation. The model may thus assist the design and tuning of the liner to achieve a standing-wave resonance with field distribution and mode spacing suitable for MRI RF excitation. The transmission properties and EM fields of a full-scale MTM lined bore for B0=4.7 T MRI are simulated including realistic losses. These methods enable robust design of thin MTM liners 30 for arbitrary bore sizes and B₀ field strengths.

Increasing magnetic field strength (B₀) has been a consistent trend in magnetic resonance imaging (MRI) due to the associated increase in image signal-to-noise (SNR). However, even at 3 Tesla (T), with a Larmor frequency of 128 MHz for standard 1H (proton) imaging, the wavelength in the high-permittivity bulk of the human body is ˜30 cm, which results in regions of signal inhomogeneity and increased local specific absorption rate (SAR) from the constructive and destructive interference of the RF fields produced during MR excitation pulses. These fields may be tuned to the Larmor frequency and provide a homogeneous, transverse, and circularly polarized magnetic field with limited local SAR.

At fields >3 T the RF inhomogeneity and local SAR constraints are limiting factors to the adoption of these field strengths for clinical use. Thus, at field strengths >3 T MRI has been largely limited to single-organ investigation using targeted or “shimmed” RF fields, or head imaging, rather than whole-body imaging. 4.7 T, the first commonly available nominal field strength higher than 3 T, may provide an SNR gain without a drastic reduction in wavelength (e.g., 7 T). The methods developed here may be applied to any field strength.

One way to improve the homogeneity of MR signal excitation and reduce the local SAR is to employ traveling waves excited by antennas placed at the ends of the MRI bore. However, there are several key drawbacks with the traveling-wave MRI approach:
1. Propagation of the MR-relevant fundamental waveguide mode (TE₁₁) only occurs above the natural cutoff frequency of the cylindrical scanner bore, which is only achievable with high field strengths (e.g., 7 T-298 MHz) and wide bores (60-70 cm diameter). Conversely, propagating modes are completely cut off in human-sized scanners operating below approximately 7 T.
2. Low transmit efficiency that must be compensated for by higher transmit power.
3. Wave reflections at air-body interfaces result in destructive interference and unpredictable regions of low signal in the body, in addition to the constructive destructive interference of standing waves in the body typical with conventional resonators.
4. Longitudinal field attenuation due to losses in the body.

A further challenge for adoption of traveling wave MRI is the dependence of the phase velocity on the MR frequency and geometry of the waveguide. Without control of the phase velocity, multiple wavelengths (analogous to longitudinal modes) may occur along the length of the bore (˜2 m) depending on the longitudinal propagation constant, resulting in nulls and hot spots, which reduce efficiency and homogeneity. Due to these drawbacks, improved SAR and homogeneity have not been achieved with basic traveling wave MRI compared to localized methods of excitation (birdcage or TEM coils).

Approaches have been proposed to address these issues. The bore can be partially filled with adjustable dielectric rods to reduce the cutoff and permit the propagation of higher-order modes, thus also providing additional degrees of freedom for field control. However, this is a complex and cumbersome method which also decreases the available bore space. Indeed, larger bore space is often needed to accommodate larger subjects, alleviate claustrophobia, and increase comfort. The use of a coaxial conductive waveguide with a central gap at the imaging region permits controllable and predictable field production and helps to focus the fields onto the imaging region, but also occupies significant space in the bore and confines the patient inside a narrow, albeit flexible, tube. Similarly, lining the surface of the bore with high-dielectric permittivity rods enhances the focusing of the field in the desired central region of the bore, reduces the attenuation in the longitudinal direction and lowers the frequency for propagation, but requires a thick liner (10 cm) and does not improve the low efficiency.

Additional modifications of the traveling-wave paradigm include ring structures that focus the field and are also attractive because the bore center is accessible. For example, the annular ladder resonator, pairs of rings made of transverse dipoles laid end-to-end or the use of “conformal resonant right-/left-handed” antennas, have all been shown to improve the centrally localized transmit efficiency of traveling wave MRI. However, the inner diameters of the rings in these three examples reduced the accessible bore diameter by 8 cm, 10 cm and 13 cm, respectively, thus occupying an impractical amount of space. Furthermore, these structures are designed for 7 T (298 MHz), and propagation of the TE₁₁ mode is already achieved with these bore diameters, so the rings are not designed to allow operation at the lower frequencies of clinical scanners, or at 4.7 T as in this study. Finally, these previous ring structures lack a complete theoretical description to guide their design, and their use has been demonstrated only with a few elements producing a limited field of view and homogeneity.

This document explores the use of metamaterials (MTMs), which are engineered structures consisting of an array of scatterers designed to produce a desired exotic macroscopic EM response for sufficiently long wavelengths. MTMs have been used in MRI to focus and control the transmit and receive RF fields. For example, to improve the localized receive sensitivity of individual elements. Transmit structures have also been developed, but comparison to established RF resonators or “coils” is still lacking.

The theoretical description of below-cutoff propagation in circular waveguides by a MTM liner with epsilon-negative and near zero (ENNZ) was first described by Pollock et al. Propagation can be far below the natural waveguide cutoff with a backwards-dispersion relation for the hybrid electric (HE) and hybrid magnetic (EH) modes (analogous to the transverse electric (TE) and transverse magnetic (TM) modes of a homogeneously filled circular waveguide). The HE₁₁ mode provides a uniform H-field conducive to MR excitation. The practical realization of an ENNZ liner was achieved by azimuthally segmented periodic ring structures with radially-directed inductors and capacitive gaps between thin-wire sections. Below-cutoff propagation was demonstrated for waveguides ˜3 cm in diameter operating at ˜3.7 GHz (approximately 43% below their natural cutoff), suggesting that such MTM liners could be scaled to permit traveling-wave MRI for fields <7 T and small bore diameters where cutoff of the TE₁₁ mode is not achieved naturally.

However, previous works did not provide a comprehensive theory of the magnetic response (characterized by the effective permeability) of the MTM structure or provide a rigorous model of how the permittivity and permeability vary with the circuit parameters, topology or geometry of the structure. Thus, this document describes a more complete effective-medium model (EMM) of the circuit layout of the MTM liner 30 rings to guide the design and optimization of the MTM liner for the MRI bore 34. It is believed that the method of analysis developed for determining the effective-medium values of a periodic ring structure has not been described previously. Unlike the previous Drude model applied for permittivity to describe all modes, the parameters derived here are spatially dispersive: the effective permeability and permittivity depend on wave-vector. Consequently, they are fundamentally nonlocal.

There are four criteria for locality: passivity, causality, absence of radiation losses and independence of the material parameters on the propagation direction. Ideally, to have physical, rather than phenomenological meaning, the constitutive parameters derived from a material homogenization method should follow the criteria for locality, but even the most commonly used homogenization methods only do so in the long-wavelength limit of propagation; these include Bloch transmission analysis, scattering parameter reflection and transmission retrieval and field averaging. Furthermore, parameter extraction in the case of anisotropic materials is ambiguous beyond very simple cases (such as from a slab of material), and results of homogenization can be highly inaccurate in the case of resonant and/or asymmetric structures, where homogenization procedures are expected to fail, or be inapplicable.

Thus, although it is desirable to characterize a metamaterial as a homogenous medium with a local constitutive relation analogous to natural materials, such a description would not accurately represent the non-local exotic electromagnetic interaction/response of many metamaterials to different excitations and across all frequencies. For this reason, the use of non-local constitutive parameters has been proposed and various methods have been established. The loss of a direct physical relation is compensated for by better predictive accuracy of the electromagnetic response of metamaterial structures. The case of multilayered dielectric media is the most well-established case where the use of non-local homogenization schemes more accurately describes the reflection/transmission from different incident angles.

The MTM liner presented in this study is a particular case where non-local constitutive parameters may be required to better characterize the electromagnetic response of the ring structure. Its circularly periodic and segmented structure results in resonant and highly non-local response, which is highly dependent on the azimuthal mode order. Thus, the EMM developed here is a non-local homogenization procedure.

The theory and design of MTM liners for MRI is disclosed herein as follows: derive an EMM of the MTM liner; compare the dispersion relation calculated using the EMM to that of the full-wave simulation; and simulation of an MTM-lined waveguide designed for MR excitation at 200 MHz (4.7 T) using the HE₁₁ mode operating as a λ/2 resonator. There is disclosed a methodological framework for designing MTM liners for MRI at any field strength.

The MTM-lined MRI bore is approximated by the structure shown in FIG. 1A and FIG. 1B, comprising a cylindrical waveguide of radius b, an accessible region with free-space permittivity (ε₀) and permeability (μ₀), and a thin interior liner of thickness tᵣ=b−a with cylindrically anisotropic material properties that take the form of diagonal tensors: ε̅ₗ=ε₀diag(εᵣ, ε_{ϕ}, ε_{z}) and μ̅ₗ=μ₀diag(μᵣ, μ_{ϕ}, μ_{z}). To generate the desired HE₁₁ mode for MR excitation it is necessary to determine the cutoffs and dispersion characteristics of this structure.

In the case of biaxial permittivity and permeability, where ε̅ₗ=ε₀diag(εᵣ, εᵣ, ε_{z}) and μ̅ₗ=μ₀diag(μ_{ϕ}, μ_{ϕ}, μ_{z}), the analysis is simplified, and the cutoffs are obtained by

$$\frac{{J}_{n}^{′} ( {k}_{0} a )}{{J}_{n} ( {k}_{0} a )} - \frac{\sqrt{{μ}_{z}}}{\sqrt{{ε}_{r}}} \frac{{G}_{n}^{′} ( {k}_{0} \sqrt{{ε}_{r} {μ}_{z}} a )}{{G}_{n} ( {k}_{0} \sqrt{{ε}_{r} {μ}_{z}} a )} = 0 , for {HE}_{n 1} modes \qquad ( A1a )$$

$$\frac{{J}_{n}^{′} ( {k}_{0} a )}{{J}_{n} ( {k}_{0} a )} - \frac{\sqrt{{ε}_{z}}}{\sqrt{{μ}_{ϕ}}} \frac{{F}_{n}^{′} ( {k}_{0} \sqrt{{ε}_{z} {μ}_{ϕ}} a )}{{F}_{n} ( {k}_{0} \sqrt{{ε}_{z} {μ}_{ϕ}} a )} = 0 , for {EH}_{n 1} modes \qquad ( A1b )$$

$$where {F}_{n} ( xa ) = {Y}_{n} ( xb ) {J}_{n} ( xa ) - {J}_{n} ( xb ) {Y}_{n} ( xa ) , and \qquad ( A1c )$$

$${G}_{n} ( xa ) = {Y}_{n}^{′} ( xb ) {J}_{n} ( xa ) - {J}_{n}^{′} ( xb ) {Y}_{n} ( xa ) , \qquad ( A1d )$$

where n is the mode order in the \hat{ϕ} direction and Jₙ and Yₙ are the n-th order Bessel and Neumann functions, respectively. The cutoff of the HEₙ₁ modes are affected only by μ_{z} and εᵣ, while those of the EHₙ₁ modes are affected only by μ_{ϕ}and ε_{z}. The complex dispersion behaviour of the EH and HE modes at frequencies away from cutoff depend on all components of the anisotropic permittivity and permeability.

A set of field equations within the liner are provided later herein for tᵣ/b<<1 (so r≈a). The fields are assumed to be homogeneous within the MTM liner region, as used in the model in the later herein. FIG. 2 shows the μ_{z} and εᵣ required for cutoff of the HEₙ₁ modes at the relevant 4.7 T ¹H MRI frequency ƒ₀=200 MHz for a=0.269 m and b=0.28 m.

Previously, radially oriented inductors and azimuthally directed wires separated by capacitive gaps were explored as a method of realizing εᵣ that is ENNZ and predict the cutoff of the HE₁₁ mode. However, a simple Drude dispersion model of the effective permittivity was applied for all modes, which accurately predicted the cutoff of the EH₀₁ mode and only approximated those of the other closely spaced HEₙ₁ modes. In the work presented here, only the HEₙ₁ (and EH₀₁) are explored, therefore an analytical model that describes μ_{z} and εᵣ with better accuracy is considered in the next section.

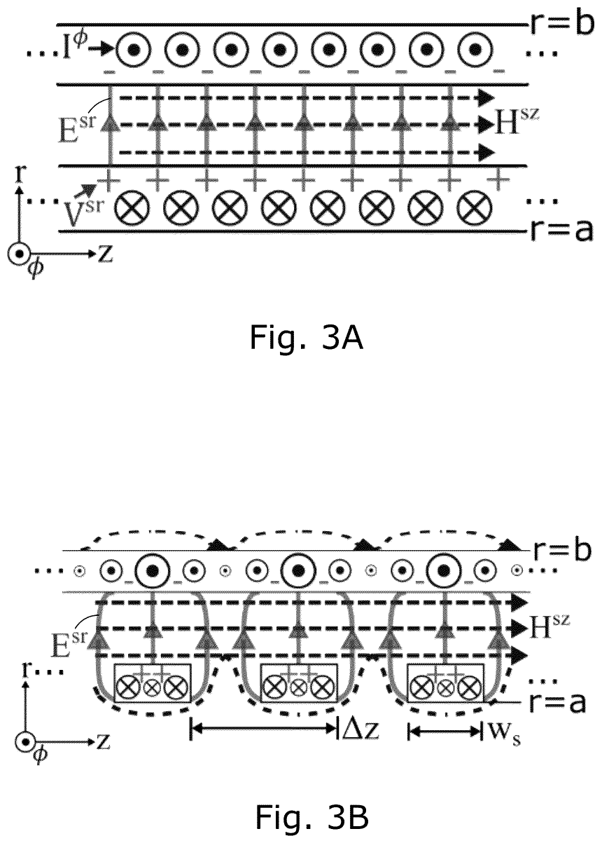

It is useful for an EMM to relate the currents/voltages on the MTM structure to the fields within the liner. Thus, to apply the derivation of the MTM-lined waveguide propagation characteristics the electromagnetic representation may describe effective ε̅ₗ and μ̅ₗ̅ that are homogeneous, with biaxial permittivity and permeability and confined to the liner region. The electrostatic solution for two layers of longitudinally infinite conducting current sheets in FIG. 3A satisfies this requirement, since the scattered radial displacement field ( [symbol] ^{sr}),

[symbol]

^{sr}=εᵣε₀ [symbol] ^{sr}=εᵣε₀V^{sr}/tᵣ\hat{r} (A2)

and the scattered longitudinal magnetizing field ( [symbol] ^{sz}),

[symbol]

^{sz}=I^{ϕ}/Δz \hat{z}, (A3)

are confined between the sheets representing the liner region, and zero outside. In this representation, both the scattered radially oriented voltage (V^{sr}) and azimuthally directed currents (I^{ϕ}) are vectors whose indices indicate the position along the azimuthal direction and are calculated for impinging HEₙ₁ fields. Thus, [symbol] ^{sr} and [symbol] ^{sz} also have an azimuthal variation as defined by the current and voltage distributions. In FIG. 3B, closely spaced conducting rings with an outer shield are represented as an approximation to the infinite current sheets in (a). This permits the calculation of the effective medium εᵣ and μ_{z} values based on the analysis of the induced currents and voltages on a single ring.

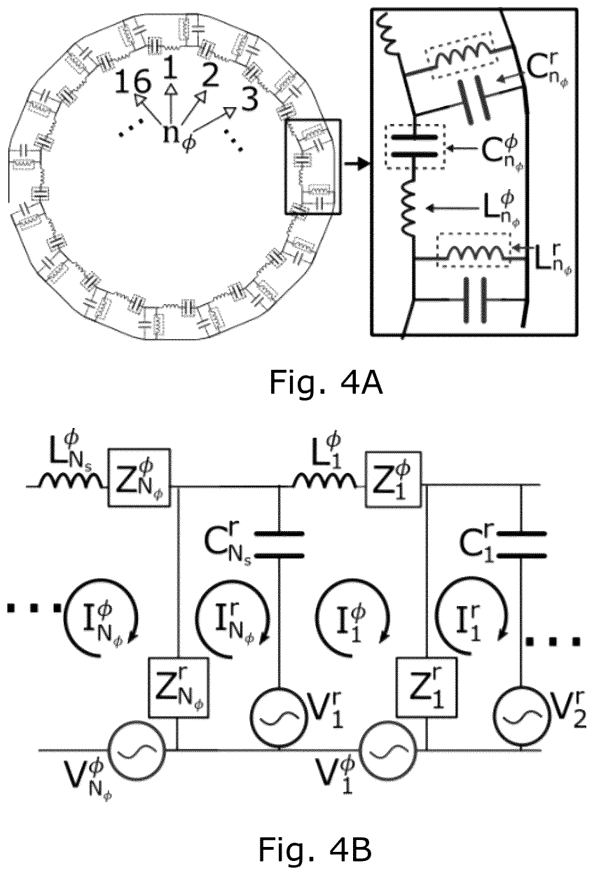

In this analysis, it is assumed that only I^{ϕ} and V^{sr} in the representation of FIG. 3 are present and longitudinally-directed currents or voltages are not induced to alter the effective medium values of μ_{ϕ}or ε_{z}, thus μ_{ϕ}=ε_{z}=1. This assumption allows the analysis of only a single ring in isolation to determine the dispersion of εᵣ and μ_{z}, as shown in FIG. 4A, in a similar manner to capacitively loaded loops or split-ring resonators. Subsequently, the process described in J. G. Pollock and A. K. Iyer, “Below-Cutoff Propagation in Metamaterial-Lined Circular Waveguides,” IEEE Transactions on Microwave Theory and Techniques, vol. 61, (9), pp. 3169-3178, 2013, which is hereby incorporated by reference in its entirety, may be used to determine the dispersion characteristics.

The ring structure is generalized to an N_{ϕ}-segment network as depicted in FIG. 4B, with the N₉₉-th and 1st segments connected. Each segment has a length of 2πa/N₉₉, with intrinsic per-unit-length series inductance (Lₙ_{_{ϕ}}^{ϕ′}) and and parallel capacitance (Cₙ_{_{ϕ}}^{r′}). Thus, each segment has an inductance and capacitance of Lₙ_{_{ϕ}}^{ϕ}=2πaLₙ_{_{ϕ}}^{ϕ′}/N_{ϕ} and C_{nϕ}^{r}=2πaC_{nϕ}^{r′}/N₉₉ , respectively (assuming short electrical length). For each segment, a lumped radial tuning impedance Zₙ_{_{ϕ}}^{r} is in parallel with Cₙ_{_{ϕ}}^{r}, and an azimuthal tuning impedance Zₙ_{_{ϕ}}^{ϕ} is in series with Cₙ_{_{ϕ}}^{r}, where n_{ϕ} is the segment number. For segments that do not include lumped impedances Zₙ_{_{ϕ}}^{ϕ}=0 and Zₙ_{_{ϕ}}^{r}=∞.

The constitutive relations for the effective permeability and permittivity of the liner link the incident fields ( [symbol] ^{iz} and [symbol] ^{ir}) and scattered fields ( [symbol] ^{sz} and [symbol] ^{sr}) to the longitudinal magnetic field ( [symbol] ^{z}) and radial electric field (E^{r}) according to

[symbol]

^{z}=μ_{z}μ₀ [symbol] ^{z}=μ₀( [symbol] ^{iz}+ [symbol] ^{sz}) (A4a)

[symbol]

= [symbol] ^{r}/εᵣε₀=( [symbol] ^{ir}+ [symbol] ^{sr})/ε₀. (A4b)

EMMs have typically been used to describe structures with unit cells (here, a single ring) with dimensions that satisfy the long-wavelength limit (dimensions <<λ). Indeed, this is the case for the radial dimensions, tᵣ, and longitudinal dimensions, γΔz, of the ring which are much less than a wavelength. To include the known angular variation the incident fields, [symbol] ^{iz} and [symbol] ^{ir} in the liner are represented by

[symbol]

^{iz}(n_{ϕ})=|H^{iz}|sin(2πn_{ϕ}n/N_{ϕ})\hat{z} (A5a)

[symbol]

^{ir}(n_{ϕ})=|D^{ir}|cos(2πn_{ϕ}n/N_{ϕ})\hat{r}. (A5b)

In these relations the continuous fields are discretized at angular positions 2πn_{ϕ}n/N_{ϕ} by the N_{ϕ} segments introduced in the circuit model. The rings are electrically large in the azimuthal direction and so the EMM modelling procedure differs from that of previous methods by considering the sinusoidal azimuthal variation of the field for the different HEₙ₁ modes. Typically, uniform plane waves have been assumed. Thus, the effective-medium μ_{z} and εᵣ are found by field averaging, also termed the field summation method, with the ratio of total fields to the incident fields in the liner region given by

$${μ}_{Z} = \frac{( {\vec{H}}^{iz} + {\vec{H}}^{sz} ) \cdot {\vec{H}}^{iz} / \vert {\vec{H}}^{iz} \vert}{{\vec{H}}^{iz} \cdot {\vec{H}}^{iz} / \vert {\vec{H}}^{iz} \vert} = 1 + \frac{{\vec{H}}^{sz} \cdot {\vec{H}}^{iz} / \vert {\vec{H}}^{iz} \vert}{{\vert {\vec{H}}^{iz} \vert}_{1}} \qquad ( A6a )$$

$${ε}_{r} = \frac{{\vec{D}}^{ir} \cdot {\vec{D}}^{ir} / \vert {\vec{D}}^{ir} \vert}{( {\vec{D}}^{ir} + {\vec{D}}^{sr} ) \cdot {\vec{D}}^{ir} / \vert {\vec{D}}^{ir} \vert} = \frac{1}{1 + {\vec{D}}^{sr} \cdot {\vec{D}}^{ir} / ( \vert {\vec{D}}^{ir} \vert {\vert {\vec{D}}^{ir} \vert}_{1} )} . \qquad ( A6b )$$

The dot product of the incident field with itself divided by its magnitude (L¹-norm) represents the field averaged across all segments of the ring. The dot product of the incident field and scattered field represents the correlation of the induced and incident fields. This accounts for the difference in phase or sign/direction of the incident vs. scattered fields along the azimuthal direction.

With the link between the incident and scattered fields and the effective medium properties established by (A6a) and (A6b), the relation between the current and voltages induced on the MTM ring structure and the scattered fields is presented as follows. The voltages induced on one azimuthal section of the ring by [symbol] ^{z}(V^{ϕ}) and [symbol] ^{r}(V^{r}) are

Vₙ_{_{ϕ}}^{ϕ=-}jωμ₀ [symbol] ₙ_{_{ϕ}}^{iz}tᵣπ(b+a)/N_{ϕ} (A7a)

Vₙ_{_{ϕ}}^{r}= [symbol] ₙ_{_{ϕ}}^{ir}tᵣ/εᵣε₀. (A7b)

Using network mesh equations, the impedance matrices describing the self-impedance of the [symbol] ^{sz}-producing meshes (Z_{H}), the self-impedance of the [symbol] ^{sr}-producing meshes (Z_{E}), and the coupling between meshes (Z_{HE}) shown in FIG. 4B are, respectively,

$${Z}_{H} ( {m}_{ϕ} , {n}_{ϕ} ) = \{ \begin{matrix}{Z}_{{n}_{ϕ}}^{ϕ} + {Z}_{{n}_{ϕ}}^{r} + j ω {L}_{{n}_{ϕ}}^{ϕ} + 1 / j ω {C}_{{n}_{ϕ}}^{r} , {m}_{ϕ} = {n}_{ϕ} \\ 0 , otherwise\end{matrix} \qquad ( A8a )$$

$${Z}_{E} ( {m}_{ϕ} , {n}_{ϕ} ) = \{ \begin{matrix}{Z}_{{n}_{ϕ}}^{r} + 1 / j ω {C}_{{n}_{ϕ}}^{r} , {m}_{ϕ} = {n}_{ϕ} \\ 0 , otherwise\end{matrix} \qquad ( A8b )$$

$${Z}_{HE} ( {m}_{ϕ} , {n}_{ϕ} ) = \{ \begin{matrix}- {Z}_{{n}_{ϕ}}^{r} , {m}_{ϕ} = {n}_{ϕ} \\ - 1 / j ω {C}_{{n}_{ϕ}}^{r} , {m}_{ϕ} = {n}_{ϕ} + 1 \\ 0 , otherwise\end{matrix} . \qquad ( A8c )$$

Because the radially-oriented impedances Zₙ_{_{ϕ}}^{r} and 1/jωCₙ_{_{ϕ}}^{r} dominate inter-mesh coupling, the intrinsic mutual impedances (i.e., mutual inductances) are neglected. The mesh currents for a given voltage excitation are then found as

$$[ \begin{matrix}{I}^{ϕ} \\ {I}^{r}\end{matrix} ] = {Z}^{- 1} V = {[ \begin{matrix}{Z}_{H} & {Z}_{HE} \\ {Z}_{HE}^{′} & {Z}_{E}\end{matrix} ]}^{- 1} [ \begin{matrix}- {V}^{r} + {V}^{ϕ} \\ {V}^{r}\end{matrix} ] , \qquad ( A9 )$$

where the prime (′) denotes Hermitian transpose.

The voltages induced across the Cₙ_{_{ϕ}}^{r} capacitances (V^{sr}) are

Vₙ_{_{ϕ}}^{sr}=(Iₙ_{_{ϕ}}^{r}−Iₙ₋₁^{ϕ})/jωCₙ_{_{ϕ}}^{r}. (A10)

Therefore, the effective-medium μ_{z} and εᵣ obtained from (A6a) and (A6b) in terms of the voltages and currents are

$${μ}_{z} = ( 1 - j \frac{ω {μ}_{0} {t}_{r} π ( b + a ) {I}^{ϕ} \cdot ( {V}^{ϕ} / \vert {V}^{ϕ} \vert )}{{N}_{ϕ} Δ z {\vert {V}^{ϕ} \vert}_{1}} ) , \qquad ( A11a )$$

$${ε}_{r} = 1 / ( 1 + ( {V}^{sr} \cdot ( {V}^{r} / \vert {V}^{r} \vert ) / {\vert {V}^{r} \vert}_{1} ) ) . \qquad ( A11b )$$

For Zₙ_{_{ϕ}}^{ϕ}=1/jωC^{ϕ} and Zₙ_{_{ϕ}}^{r}→∞ the induced currents I^{ϕ are}

I^{ϕ}=−jωμ₀tᵣπ(b+a) [symbol] ^{iz}/(jωL^{ϕ}+1/jωC^{ϕ})N_{ϕ} (A12)

And (A11a) simplifies too

$${μ}_{z} = ( 1 - \frac{{πμ}_{0} {t}_{r} ( b + a )}{Δ {zN}_{ϕ} {L}^{ϕ} ( 1 - {ω}_{0 m}^{2} / {ω}^{2} )} ) , \qquad ( A13 )$$

where the resonance frequency of ϕ-directed impedances is ω₀ₘ=1/\sqrt{L^{ϕ}C^{ϕ}}. This form is comparable to that of a split-ring resonator. For Zₙ_{_{ϕ}}^{ϕ}→∞ and Zₙ_{_{ϕ}}^{r}=jωL^{r}

I^{r}=V^{r}/(jωL^{r}+1/jωC^{r}), (A14)

and (A11b) simplifies to

εᵣ=(1−ωₚ²/ω²). (A15)

with plasma frequency ωₚ=1/\sqrt{L^{r}C^{r}}, which is approximately equal to the EH₀₁ mode cutoff. Equation (A15) is the Drude dispersion without damping, which describes the permittivity dispersion of a metallic 3D lattice microstructure comprising a regular array of thin wires. Thus, (A13) and (A15) show that the εᵣ of each mode will follow a Drude dispersion and μ_{z} will be near unity if C^{ϕ}→0, so Zₙ_{_{ϕ}}^{ϕ}→∞ and ω₀ₘ→∞, which was assumed in previous works.

The intrinsic inductance per segment for both cases was set as L^{ϕ}=40 nH. However, to better match the results of full wave simulation of the dispersion the intrinsic radial capacitance was slightly different for the two cases: C^{r}=1.77 pF for the MNL case and C^{r}=1.43 pF for the ENNZ case. The voltage and current distributions of the ENNZ and MNL cases will differ to some extent, so the circuit equivalent values in the EMM are expected to differ slightly.

For the two cases, FIG. 5 shows the effective-medium values calculated from Eqs. (A11a) and (A11b), and corresponding HEₙ₁ cutoff from (A1a). For the MNL case, when n>0 the cutoffs occur at frequencies below the μ_{z} poles (where εᵣ=0). For n=0, the cutoff occurs higher in frequency than the μ_{z} pole, here both μ_{z} and εᵣ are non-zero and negative (see FIG. 2). For the ENNZ case the cutoffs occur when μ_{z} is positive and much smaller in magnitude (<4) compared to the MNL case. Furthermore, the cutoffs occur when εᵣ=0 for the EH₀₁ case, and when εᵣ is negative and near zero for the n>0 cases.

The novelties of this analysis compared to previous work include the description of the MNL case, and the need for different Drude-like permittivity responses to accurately predict the cutoffs for each ENNZ mode. Furthermore, the effective μ_{z} was assumed to be near unity in the ENNZ case. Finally, because [symbol] ^{ir} and [symbol] ^{iz} depend on n (see (A5)), so will μ_{z} and εᵣ defined in (A11a/b) and the EMM is non-local. In the case presented here, the non-local constitutive parameters converge to local ones in the long-wavelength limit: the constitutive relations for the EH₀₁ and HE₀₁ modes fit this criterion and are approximated by the Lorentz-type permeability and Drude-type permittivity relations given in Eqs. (A13) and (A15), respectively. However, these approximations are of limited use in the derivation of the higher order mode cutoffs and propagation curves, such as the HE₁₁ mode for operation in this study.

How many segments the cylindrical geometry is divided into is another consideration in deriving constitutive parameters for the liner region (8 segments here). Due to the cylindrical geometry and the N_{seg} periodicity, only integer values of the azimuthal mode orders up to half that of the number of segments are allowed, and therefore the derived constitutive parameters are valid only for those values. The waveguide can only support electromagnetic propagation with those azimuthal variations and the response to impinging fields will be a linear combination of the response of the allowable azimuthal modes.

A criterion used herein for the applicability of an examplary EMM model follows that stated in C. R. Simovski, “Material parameters of metamaterials (a Review),” Optics and Spectroscopy, vol. 107, (5), pp. 726, 2009. “The homogenization has sense if its result (the effective material parameters) can be used for solving boundary problems of electrodynamics where a discrete set of scattering particles is replaced by a sample of a continuous medium.” Thus, the constitutive parameters derived here (μ_{z} and μᵣ) have the following limited definition: the replacement of the region composed of the space between MTM rings and outer conductor with a homogeneous material, with the constitutive parameters derived, can be used in the analytical boundary solution of the geometry shown in FIG. 1A. Thus, the propagation constant and the surface-averaged E-field and H-field can be approximated by the boundary problem solution.

With the EMM developed, the methods of J. G. Pollock and A. K. Iyer, “Below-Cutoff Propagation in Metamaterial-Lined Circular Waveguides,” IEEE Transactions on Microwave Theory and Techniques, vol. 61, (9), pp. 3169-3178, 2013, which is hereby incorporated by reference in its entirety, can be used to determine the propagation more accurately; this will be demonstrated in the next section by comparison to full-wave simulation. The validity of this procedure may be expected to be limited to the prediction of the cutoffs and k_{z} near-zero propagation. Its usefulness is highlighted by the resulting close match of the behaviour of the dispersion curves for a large range of frequency when applying the analytical solution with derived bulk constitutive parameters.

The changes in dispersion, cutoff and mode spacing as a function of the equivalent circuit model parameters are demonstrated in the supplementary material. This section covers the determination of the EMM parameters to match dispersion and cutoffs obtained from full-wave analysis of the MTM liner structure. The objective is to find a design where the phase shift produces a −λ/2 resonance along a length of MTM-lined MRI bore 34, so that the extent of the magnetic field produced is comparable to the imaging field-of-view of the scanner (˜25 cm). For a typical bore length of 1-2 m only a portion of the bore would include the liner. An 80 cm length of liner is used here, which is near the free-space λ/2=0.75 m for 4.7 T (200 MHz).

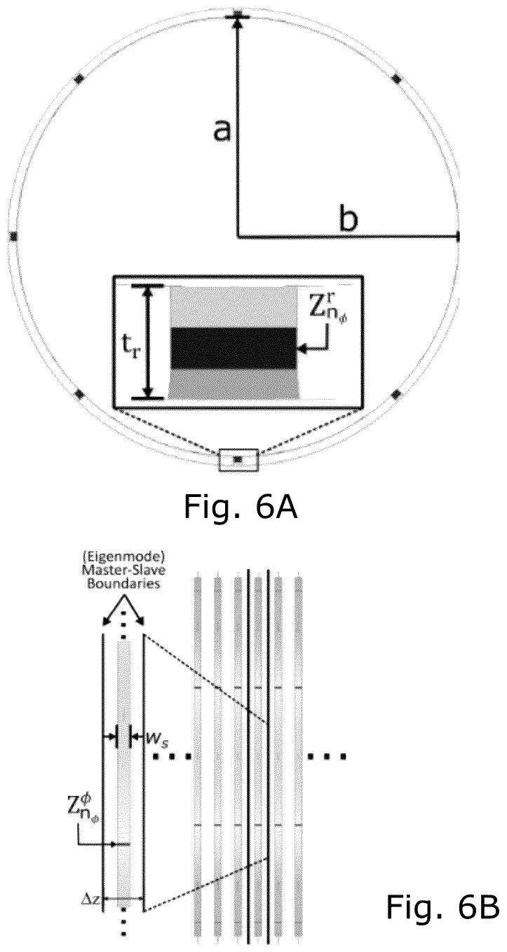

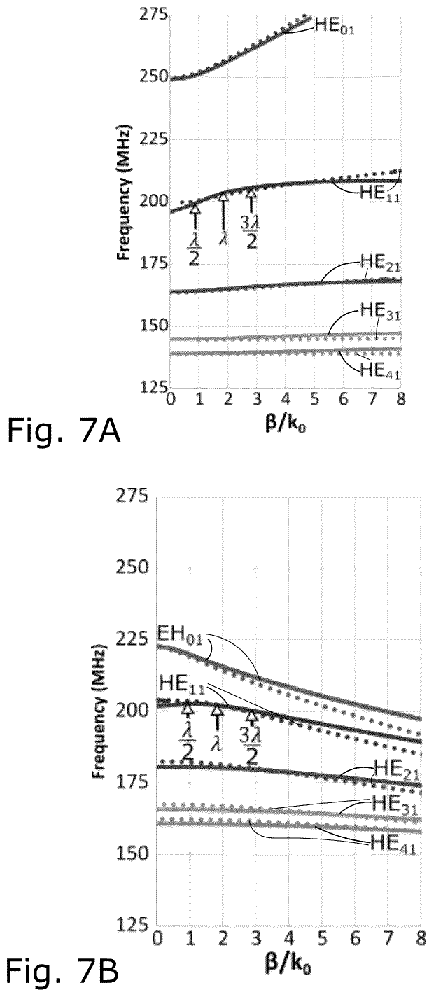

The eigenmode simulator in HFSS (Ansys, Canonsburg, PA) was used to find the dispersion curves. A single ring of longitudinal extent Δz is used as a unit cell with master-slave boundaries on the faces of the thin cylindrical volume as labelled in the structure in FIG. 6B. The eigenmode frequencies were solved for each π/60 phase step between faces (FIG. 7).

The thin conductive strips making up the rings are backed by the conductive shield of the waveguide or bore, much like microstrip lines, but the fields are altered due to the presence of the adjacent rings. Nevertheless, for the EMM to correctly model the structure in FIG. 6, the values of C^{r} and L^{ϕ} are expected to be between those calculated for biplanar lines and those calculated for a microstrip segment of 2πa/N_{ϕ}=7.04 cm length. Thus, calculated from quasistatic models we would expect 31.0 Nh<L^{ϕ}<97.3 Nh (microstrip<biplanar lines) and 0.57 pF<C^{r}<1.78 Pf (biplanar lines<microstrip). Considering this appreciable range, the models were tuned by first adjusting L^{ϕ} so the HE₀₁ mode cutoffs matched in the MNL cases, then C^{r} adjusted so the EH₀₁ mode cutoffs matched in the ENNZ cases.

The C^{ϕ} capacitor values and L^{r} inductor were 0.25 pF and 2 nH lower for the full-wave simulation than the values used for the EMM, respectively. The values are higher in the EMM to account for the small additional stray capacitance introduced by the physical gap between conductors where the C^{ϕ} capacitors are, and to account for the small inductance of the radial galvanic connection between the rings and the outer MRI shield/bore. These two parameters affect the spacing of modes.

Therefore, the simulation parameters of the two cases are summarized as follows:
FIG. 7A—MNL, C^{ϕ}=2.9 pF, L^{r}=50 nH
FIG. 7B—ENNZ, C^{ϕ}=0.5 pF, L^{r}=108 nH

Relating the EMM-predicted dispersion in FIG. 7 to effective medium values in FIG. 5 demonstrates that the MNL is forward propagating (i.e., with parallel phase and group velocities), while the ENNZ is, as observed in previous studies, backwards propagating (with antiparallel phase and group velocities).

Based on the close agreement between mode cutoffs predicted from Eigenmode simulation and the EMM framework developed here, the MTM liner can consequently be designed efficiently using the EMM with accurate calculations of only a few readily obtained geometrically derived impedances in the ring structures.

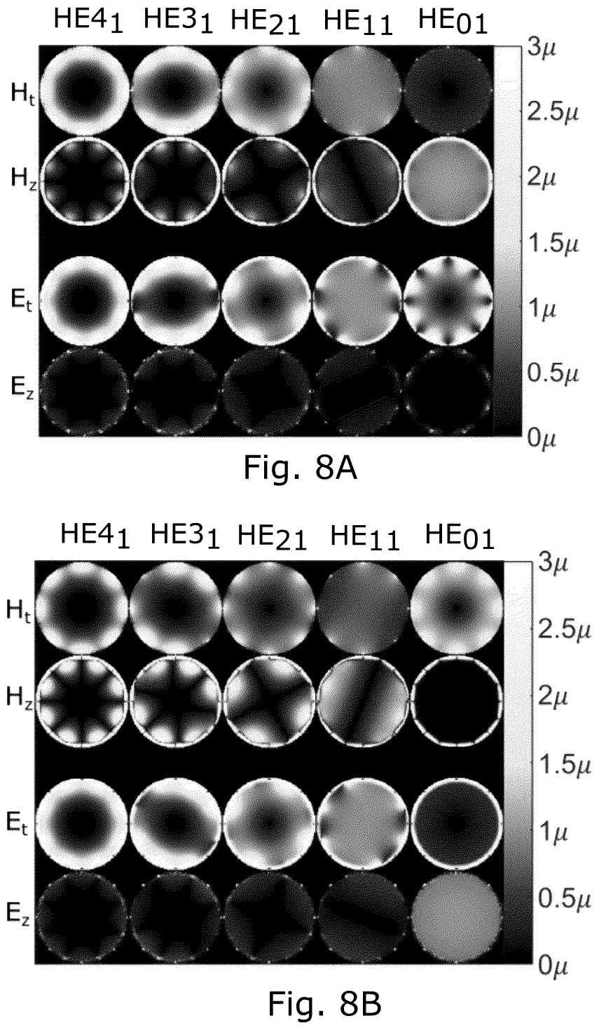

The bilayer infinite current sheet representation is a simple and effective model because the fields induced by the transverse currents are confined to the liner region and are directly related to the currents and voltages. However, capacitive coupling between rings introduces longitudinal currents, so fields are not strictly confined to the liner region. This is a cause of some of the (minor) discrepancy in the analytical vs. simulated dispersion in FIG. 7. Additionally, a biaxial permittivity has been assumed example calculations herein, ε̅ₗ=ε₀diag(εᵣ, εᵣ, 1), which limits the accuracy of the EMM developed here: the radial permittivity (εᵣ\hat{r}) is found and assigned equally to the azimuthal permittivity (ε_{ϕ}\hat{ϕ}). The effect of this assumption is demonstrated in the later herein, by comparing the dispersions obtained in three cases: ε̅ₗ=ε₀diag(εᵣ, εᵣ, 1), ε̅ₗ=ε₀diag(εᵣ, 1,1) and ε̅ₗ=ε₀diag(1, εᵣ, 1). The results suggest that ε_{ϕ}\hat{ϕ} has a minimal effect on the dispersion and so the biaxial permittivity approximation is valid for describing the MTM liner. A fully descriptive EMM may consider the full anisotropic material property tensors including the impact of longitudinal currents and coupling between rings. The issue of accounting for coupling between rings and longitudinal currents is explored more fully with the network model disclosed later herein. The simulated electric and magnetic fields for 1 J of energy stored in each mode are shown in FIG. 8. From these field plots it is observed that only the HE₁₁ mode produces the uniform transverse H-field required for MR excitation. There are observable differences in the field profiles of the MNL or ENNZ cases due to the slightly different induced current expected from Eq. (A9) because of the different impedance distributions along the ring. As expected, strong transverse electric fields and longitudinal magnetic fields are observed in the liner region, which are accounted for in the EMM by the effective εᵣ near zero and large μ_{z}, respectively.

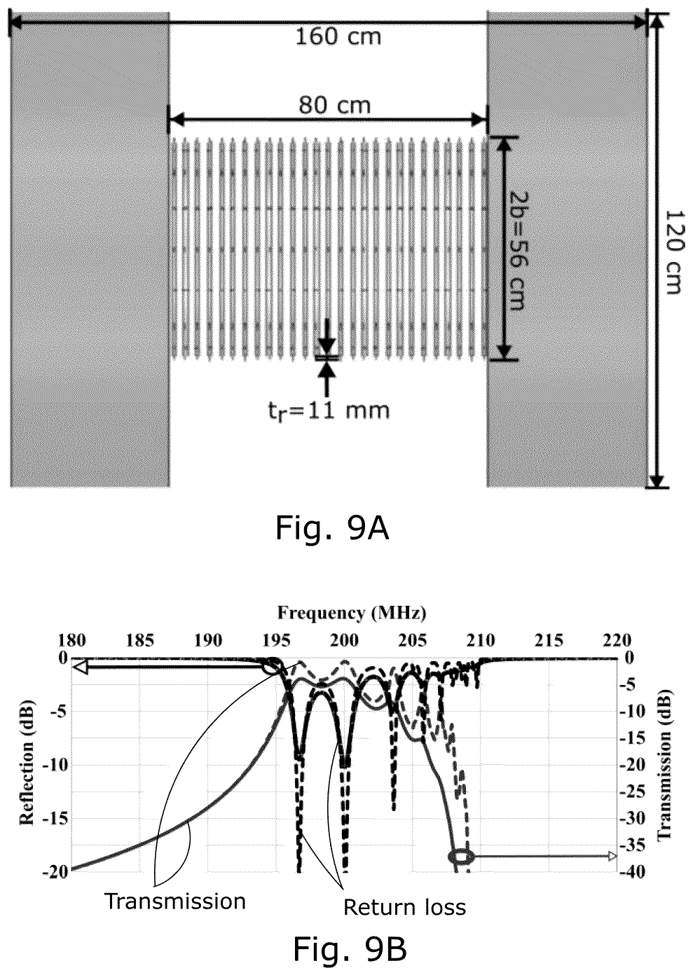

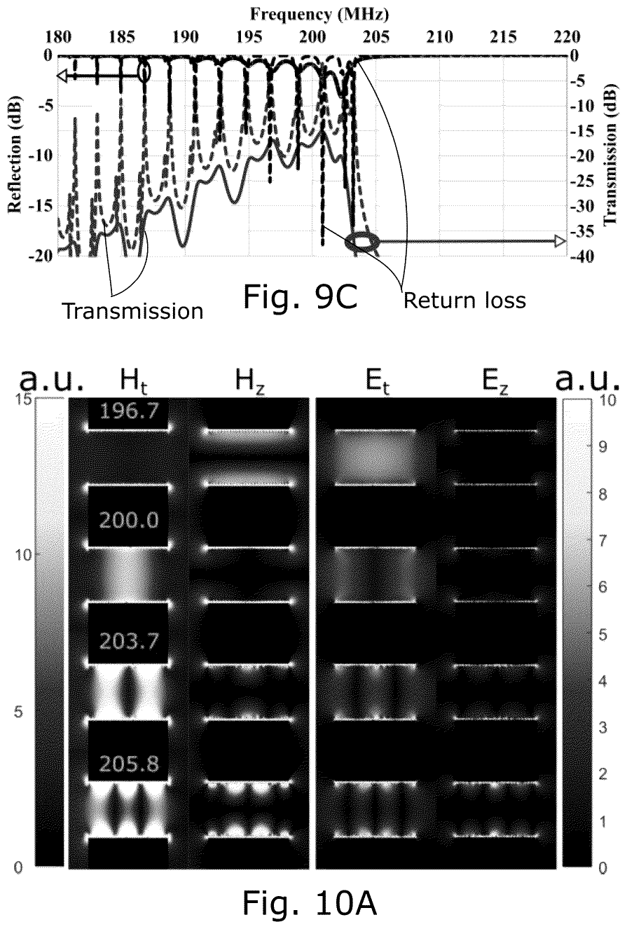

From either the EMM or full-wave eigenmode simulation the predicted frequency for a desired λ/2 longitudinal resonance mode along a waveguide is indicated by labelled arrows on the dispersion graphs (FIG. 7). Thus, a full-scale liner consisting of N_{z}=27 rings was simulated with the dimensions shown in FIG. 9A, including an MTM-lined section of waveguide suitable for providing body MR excitation at 4.7 T (200 MHz). The radius of the waveguide matches that of the 4.7 T MR bore (1.72 m length and 0.28 m radius) available for future testing at the University of Alberta. A λ/2 resonance is predicted at ˜200 MHz for the cases presented in FIG. 7. The lined section is placed between two larger-radius vacuum-filled waveguides, with PEC boundaries on outer faces, that are above cutoff for the HE₁₁ (TE₁₁) mode to couple the lined waveguide to the simulation ports. The two above-cutoff waveguides are fed by TE₁₁ wave ports and thus only HE₁₁ propagation is investigated here. Loss in the copper rings and outer RF shield of the MRI scanner 32 was included (conductivity of 5.8×107 S/m). To further model realistic losses, the liner's conductors included a surface roughness of 1 μm and the lumped elements were assigned realistic Q factors at 200 MHz (for the MNL case, C^{ϕ}=2.9 pF with Q=1000 and L^{r}=50 Nh with Q=150; for the ENNZ case, C^{ϕ}=0.5 pF with Q=1000 and L^{r}=108 nH with Q=200). The transmission and reflection parameters of the uniform mode (HE₁₁) for the MNL and ENNZ cases are shown in FIG. 9B and FIG. 9C, respectively. The bandwidth and magnitude of the transmission peaks are smaller for the ENNZ case compared to the MNL case. When realistic losses are included the transmission decreases more for the ENNZ case due to the larger, lossy radial inductors. Analyzing the complete fullscale liner presented here using the full-wave simulation method requires extensive simulation time with a dedicated 1 TB RAM server. The simulation was used here to benchmark the eigenmode (>15 GB RAM and >5 min per point) and EMM methods (<1 GB RAM and <0.5 min per point) which are computationally orders of magnitude more efficient.

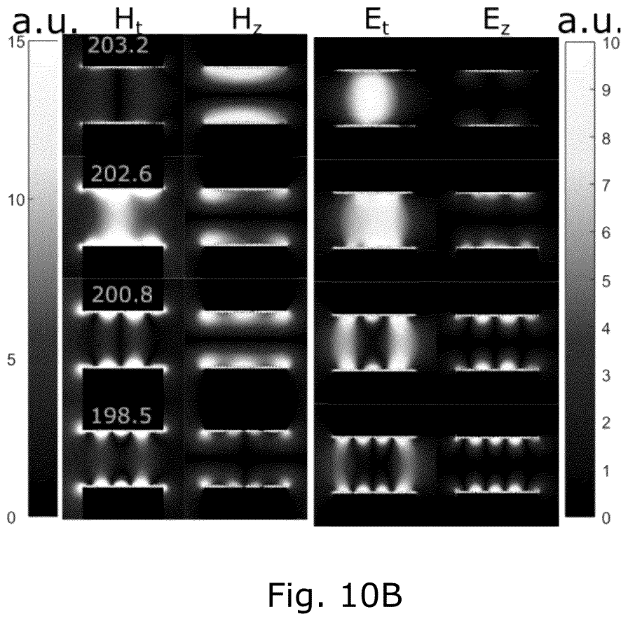

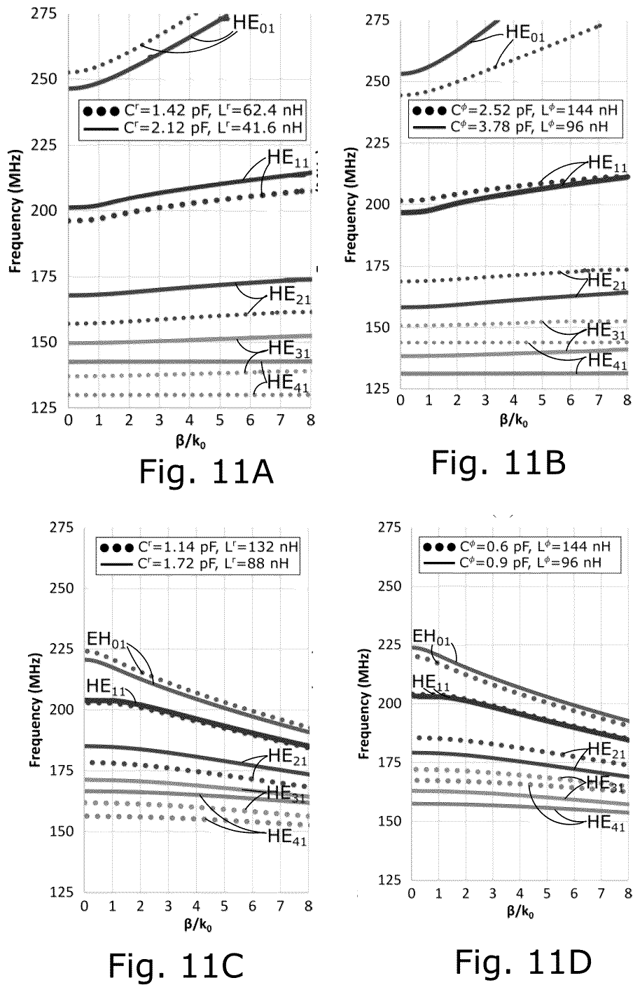

The transverse and longitudinal E and H fields for key resonances/dispersion values (cutoff, λ/2, λ and 3 λ/2) are shown in FIG. 10. For the MNL case distinct longitudinal modes are observed, while for the ENNZ case a mixing of longitudinal modes is observed. For example, there is a strong null in the center of the waveguide for the transverse magnetic field of the cutoff resonance, like that of the A resonance. This mode mixing results from the ENNZ case flat dispersion curve (FIG. 7B).

The proposed exemplary MTM liner is tuned for the HE₁₁ λ/2-resonance (2^{nd} mode in both FIGS. 10A and 10B), which satisfies the requirements of an effective MR transmit resonator by producing a strong and homogeneous transverse field in the center of an MR-bore sized waveguide. Furthermore, the HE₁₁ mode can be driven in quadrature, for more efficient circularly-polarized MR excitation. The newly developed MNL, and existing ENNZ case were compared, and the less desirable properties of the ENNZ case—i.e., more extreme dispersion, smaller transmission bandwidths, mixing of modes, need for large lossy inductors and strong dependence on a parasitic capacitance—favor the MNL case for practical MTM liner designs for MRI. The transmission performance metrics of a similarly-designed MNL-case MTM liner were compared to those of the conventional birdcage coil at 3 T and 4.7 T, demonstrating equivalent transmit efficiency with better RF safety (lower specific absorption rate).

The MTM liner 30 is related to conventional resonators in MRI in some respects. For example, the frequency response of the birdcage coil and TEM resonator also have distinct n-th order azimuthal modes based on the number of rungs/legs of the resonators, which is analogous to the n-th order of the HEₙ₁ modes. Additionally, birdcage coils demonstrate increasing or decreasing mode order with frequency depending on whether the configuration is low-pass or high-pass, respectively. Similarly, the MTM liner shows the same behaviour in the MNL case if the radial lumped components are inductive (low-pass) or capacitive (high-pass). In contrast, due to the longitudinal arrangement of rings the finite length MTM liner exhibits longitudinal resonances based on the number of rings. Furthermore, the currents of the MTM liner HE₁₁ mode are largely azimuthal, while the field-producing currents of the birdcage coil and TEM coil are longitudinal (more akin to the EH₁₁ mode).

The design of a full-scale MTM-lined waveguide has been demonstrated based on two concordant methods of analysis: effective medium analysis with circuit equivalents and fullwave simulation. The EMM developed provides a theoretical and conceptual framework for interpreting and understanding the operation of the MTM liner designed here for MRI excitation. The method of EMM derivation presented here is applicable to MRI bore liners for various field strengths and bore sizes, and may also be applicable to other MTM structures.

While the models are valuable design tools, full-wave simulation may still be required to determine the MRI performance metrics as well as to verify the final design. Comparison of the MTM liner approach to conventional methods of MRI excitation by a birdcage coil and evaluating the impact of realistic body loading is disclosed later herein.

In the exemplary EMM model developed the fields are approximated as radially homogeneous within the liner

$$( \frac{\partial {E}_{z}}{\partial r} = 0 , \frac{\partial {H}_{z}}{\partial r} = 0 )$$

which occurs in the limit of tᵣ<<b. Therefore, with the homogeneous field assumption, and setting r≈a, the field equations are simplified to

$${\vec{E}}_{z} = \vert {E}_{z} \vert cos ( n ϕ ) {e}^{- γ z} \qquad ( B1a )$$

$${\vec{E}}_{r} = - \frac{j ω n {μ}_{0}}{{( {γ}_{r}^{{ε}_{r}} )}^{2} a} \vert {H}_{z} \vert cos ( n ϕ ) {e}^{- γ z} \qquad ( B1b )$$

$${\vec{E}}_{ϕ} = \frac{γ n}{{( {γ}_{r}^{{ε}_{r}} )}^{2} a} \vert {E}_{z} \vert sin ( n ϕ ) {e}^{- γ z} \qquad ( B1c )$$

$${\vec{H}}_{z} = \vert {H}_{z} \vert sin ( n ϕ ) {e}^{- γ z} \qquad ( B1d )$$

$${\vec{H}}_{r} = - \frac{j ω n {ε}_{0} {ε}_{r}}{{( {γ}_{r}^{{ε}_{r}} )}^{2} a} \vert {E}_{z} \vert sin ( n ϕ ) {e}^{- γ z} \qquad ( B1e )$$

$${\vec{H}}_{ϕ} = \frac{γ n {ε}_{r}}{{( {γ}_{r}^{{ε}_{r}} )}^{2} a} \vert {H}_{z} \vert cos ( n ϕ ) {e}^{- γ z} where , \qquad ( B1f )$$

$$\vert {E}_{z} \vert = {C}_{1} {J}_{n} ( {γ}_{r}^{{ε}_{0}} a ) , \vert {H}_{z} \vert = {C}_{2} {J}_{n} ( {Y}_{r}^{{ε}_{0}} a ) and \qquad ( B1g )$$

$${γ}_{r}^{{ε}_{r}} = \sqrt{{γ}^{2} + {k}_{0}^{2} {ε}_{r} {ε}_{0} {μ}_{0}} , {γ}_{r}^{{ε}_{0}} = \sqrt{{γ}^{2} + {k}_{0}^{2} {ε}_{0} {μ}_{0}} . \qquad ( B1h )$$

C₂ and C₁ are constants, whose ratio is related by the continuity of the tangential electric and magnetic fields at r=a by

$$\frac{{C}_{1}}{{C}_{2}} = {\frac{j ω n {ε}_{0}}{γ a} [ \frac{{ε}_{r} {ε}_{0} {μ}_{0}}{{( {γ}_{r}^{{ε}_{r}} )}^{2}} - \frac{{ε}_{0} {μ}_{0}}{{( {γ}_{r}^{{ε}_{0}} )}^{2}} ] [ \frac{{J}_{n}^{′} ( {γ}_{r}^{{ε}_{0}} a )}{{γ}_{r}^{{ε}_{0}} {J}_{n} ( {γ}_{r}^{{ε}_{0}} a )} - \frac{\sqrt{{ε}_{r}} {F}_{n}^{′} ( \frac{{γ}_{r}^{{ε}_{r}} a}{\sqrt{{ε}_{r}}} )}{{γ}_{r}^{{ε}_{r}} {F}_{n} ( \frac{{γ}_{r}^{{ε}_{r}} a}{\sqrt{{ε}_{r}}} )} ]}^{- 1} \qquad ( B2a )$$

$$\frac{{C}_{1}}{{C}_{2}} = {\frac{j ω n {ε}_{0}}{γ a} [ \frac{{ε}_{r} {ε}_{0} {μ}_{0}}{{( {γ}_{r}^{{ε}_{r}} )}^{2}} - \frac{{ε}_{0} {μ}_{0}}{{( {γ}_{r}^{{ε}_{0}} )}^{2}} ] [ \frac{{J}_{n}^{′} ( {γ}_{r}^{{ε}_{0}} a )}{{γ}_{r}^{{ε}_{0}} {J}_{n} ( {γ}_{r}^{{ε}_{0}} a )} - \frac{\sqrt{{μ}_{z}} {G}_{n}^{′} ( \sqrt{{μ}_{z}} {γ}_{r}^{{ε}_{r}} a )}{{γ}_{r}^{{ε}_{r}} {G}_{n} ( \sqrt{{μ}_{z}} {γ}_{r}^{{ε}_{r}} a )} ]}^{- 1} \qquad ( B2b )$$

Thus, for the stated assumptions, each of the field components within the liner can be approximated by a propagating wave (e^{-γz}) with sinusoidal azimuthal variation, as in the effective medium homogenization method employed.

The impact that variations of the different parameters in the EMM (Zₙ_{_{ϕ}}^{ϕ}, Zₙ_{_{ϕ}}^{r}, Lₙ_{_{ϕ}}^{ϕ}, Cₙ_{_{ϕ}}^{r}) have on the cutoff frequencies, dispersion or mode spacing is not immediately apparent. It is desirable to have the azimuthal modes spaced as far apart as possible to avoid mode mixing. Similarly, it is also desirable for the slope of the dispersion to be as large as possible so longitudinal modes are not excited simultaneously. In fact, for the purpose of MRI excitation only a finite length of MTM lined waveguide will be used, which will be comparable in length to the imaging region size of the scanner (e.g., a sphere 40-50 cm in diameter). Longitudinal resonances will occur when the length of the metamaterial liner is integer multiples of a half-wavelength. Therefore, it is desirable that the half-wavelength resonance useful for MR excitation be sufficiently separated in frequency from the other longitudinal resonances. Otherwise, with the low Q of body-loaded MRI, multiple longitudinal resonances would be excited simultaneously, leading to wavelength interference and degraded field homogeneity. This is demonstrated herein for the ENNZ case.

To evaluate the impact of the EMM impedance parameters we focus our analysis to the MNL and ENNZ cases disclosed prior. The MTM liner will be designed so that the Larmor frequency of the ¹H nucleus for the specified MRI field strength will be near the HE₁₁ cutoff frequency to favour this mode. While varying parameters for the two cases we ensure that the ω₀ₘ and ωₚ remain the same, so that the cutoff frequencies of the HE₀₁ and EH₀₁ modes, as well as the cutoff frequency of the HE₁₁ mode, change only slightly. Thus, the dispersion was calculated for the MNL and ENNZ cases with azimuthal and radial capacitances:
MNL case:
(C^{ϕ}=2.52 pF, L^{ϕ}=144 nH),
(C^{ϕ}=3.78 pF, L^{ϕ}=96 nH),
(C^{r}=1.42 pF, L^{r}=62.4 nH),
(C^{r}=2.12 pF, L^{r}=41.6 nH),
ENNZ case:
(C^{ϕ}=0.6 pF, L^{ϕ}=144 nH),
(C^{ϕ}=0.9 pF, L^{ϕ}=96 nH),
(C^{r}=1.14 pF, L^{r}=132 nH),
(C^{r}=1.72 pF, L^{r}=88 nH),

The dispersion curves in FIG. 6 were calculated using the analytical equations for the complex propagation constant (γ=a+jβ) of an MTM lined circular waveguide (only β is shown), with the εᵣ and μ_{z} calculated from the EMM model. Smaller C^{r} (FIG. 6A and FIG. 6C) and larger C^{ϕ} (FIG. 6B and FIG. 6D), result in greater mode spacing when L^{r} and L^{ϕ} are tuned to keep the mode cutoff approximately the same. Overall, the impact on the dispersion slope is limited for the parameters evaluated; only the larger C^{r} for the MNL case (FIG. 6B) results in an appreciably larger dispersion slope. It is expected that the coupling between rings, impacting the other elements of ε̅ₗ and μ̅ₗ, will affect the dispersion farther from cutoff to a greater extent, but this is not accounted for in the model presented here.

The L^{r} and C^{ϕ} parameters represent lumped impedances that would be realized by lumped capacitors or inductors, and thus can be readily tuned. In contrast, the C^{r} and L^{ϕ} parameters are dependent on the MTM liner structure and can only be changed by altering the geometry. For example, thinner conductors would reduce C^{r} and increase L^{ϕ}, which, however, leads to opposing effects for achieving the desired increase in mode spacing.

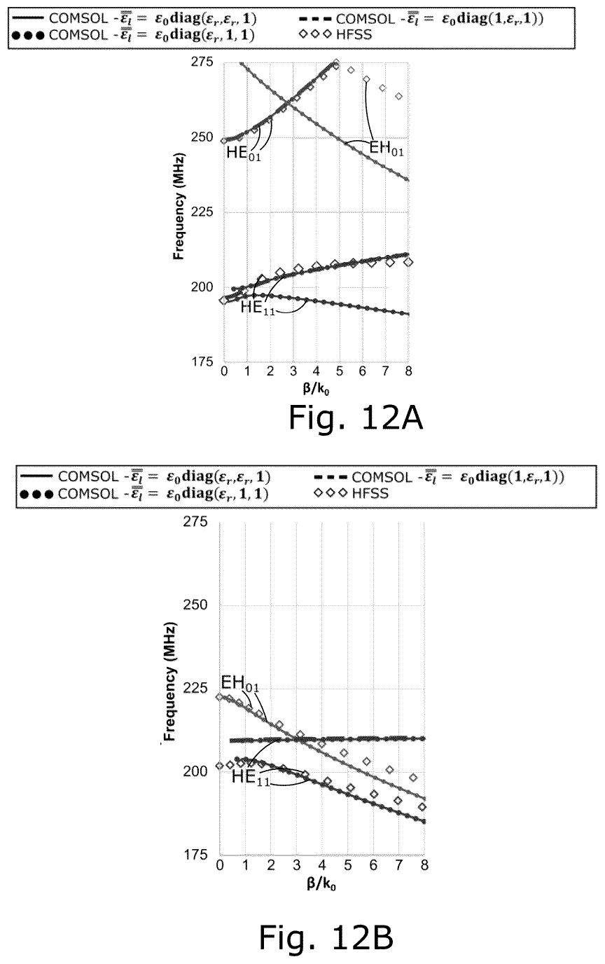

In exemplary calculations presented here, the biaxial approximation |ε_{ϕ}\hat{ϕ}|=|εᵣ\hat{r}| was made, so ε̅ₗ=ε₀diag(εᵣ, εᵣ, 1). To test this assumption, in conjunction with the developed EMM, the dispersion for three different cases is compared in FIG. 12: ε̅ₗ=ε₀diag(εᵣ, εᵣ, 1), ε̅ₗ=ε₀diag(εᵣ, 1,1) or ε̅ₗ=ε₀diag(1, εᵣ, 1). The dispersion curves shown for the three cases were simulated with the COMSOL eigenmode solver using the MTM liner geometry shown in FIG. 1A. The same effective medium and geometric parameters were used as those for FIG. 7. The simulated values obtained using COMSOL were found to closely match the analytically derived values, making these results indistinguishable. Therefore, only the simulated values are included. Additionally, only the HE₁₁, HE₀₁ and EH₀₁ modes are shown, as the behavior of the additional HEₙ₁ modes closely resembles that of the HE₁₁ mode, but shifted in frequency. For context, the HFSS simulated values of the practically realized structure are included.

The modes' cutoff frequencies may be more sensitive to {εᵣ\hat{r}} than to {ε_{ϕ}\hat{ϕ}}. Due to this weak dependence on {ε_{ϕ}\hat{ϕ}}, for the remainder it is assumed that a uniaxial transverse permittivity.” From FIG. 12 it is observed that the cut-off and mode propagation are highly dominated by εᵣ\hat{r} compared to ε_{ϕ}\hat{ϕ}.

The EH₀₁ mode, which is only facilitated by the ENNZ property, does not exist for the ε̅ₗ=ε₀diag(1, εᵣ, 1) case and thus depends only on the radial permittivity. Furthermore, the backwards propagating ENNZ regions are not present for the HEₙ₁ modes if ε̅ₗ=ε₀diag(1, εᵣ, 1). Additionally, the forward propagating regions, where epsilon is positive and near zero, are present if ε̅ₗ=ε₀diag(εᵣ, 1,1), while the backwards propagating region in the MNL case does not occur if ε̅ₗ=ε₀diag(1, εᵣ, 1). However, this latter feature also occurs with the practically realized MTM liner as simulated with HFSS.

Ultimately, the EMM model developed here only explicitly derives a value for εᵣ\hat{r}, while the value of ε_{ϕ}\hat{ϕ} is found to be largely inconsequential. Therefore, since applying the uniaxial transverse permittivity approximation has no significant effect on the analytically derived dispersion values, and due to its usefulness in simplifying the concept and application of the analytically derived MTM liner equations, it is still used in this work. However, the backwards propagating ENNZ region for the MNL case, and the forward propagating epsilon positive and near zero region (with flat slope, so highly dispersive) for the ENNZ case, are not included in our comparisons of the HEₙ₁ modes, since are not observed to have a corresponding relation with the HFSS simulated MTM liner (both the eigenmode and full MTM-lined bore models), where simulation is expected to match real-world implementation.

There is disclosed a metamaterial liner that may be employed as a resonator in MRI and design methods similar to conventional resonators for MRI can be used. A network model that accounts for coupling between the rings of the metamaterial liner structure is developed, including axial connections and direct port excitation. The model is tested by comparing simulation of the metamaterial liner dispersion characteristics, the input impedance and predicted resonances and currents. To test its application for MRI, the transmit performance in the body is compared to a conventional birdcage coil (4.7 T-200 MHz) demonstrating benefits.

It is demonstrated how the example liner can be described in terms of a resonator using only circuit/network theory as opposed to computationally expensive simulations. The optimization/tuning and variation of the lumped impedance parameters is also presented.

A design method is disclosed for the metamaterial liner as a transmit resonator for MR (facilitating a traveling wave type excitation) applicable to any field strength, which has not been simulated or presented prior to our work. Methods of analyzing the novel metamaterial liner are developed. The performance metrics of a practical design are quantified and compared at 4.7 T, demonstrating that the metamaterial liner has greatly reduced localized SAR (power deposition into tissues) for the same RF magnetic transmit field compared to the conventionally used birdcage coil design. This provides evidence that the metamaterial liner 30 should replace the birdcage coil in future MRI scanner designs, as well as a retrofit to existing scanners.

The theoretical foundations of a metamaterial (MTM) liner for the MRI bore, that facilitates the propagation of reduced-cutoff cylindrical waveguide modes, are presented above. The practical design and modelling of the novel metamaterial liner applied as a body MRI radio-frequency (RF) transmitter at 4.7 T is disclosed. An equivalent network mesh model is developed to reduce the distributed structure of the MTM to one that uses lumped discrete tuning elements. The close match between full-wave simulation and the network model is demonstrated by comparison of the longitudinal propagation dispersion curves and input impedance. The application of the methods developed for analysis of the MTM liner behavior are demonstrated in the design of a practical MTM liner as an effective MRI RF transmitter. The liner's simulated performance was evaluated using three key metrics of MR radiofrequency (RF) coil performance: transmit efficiency, homogeneity of the transmit field, and the 10 g averaged local specific absorption rate (SAR). These metrics are compared to those for a commonly used birdcage (BC) coil. The main advantage of the MTM liner is the 50% reduction in maximum SAR relative to the BC coil, with comparable transmit efficiency and homogeneity. Thus, the liner can replace the BC coil as an RF transmitter and provide an enhanced safety margin, or allow the use of faster, more SAR-aggressive imaging sequences.

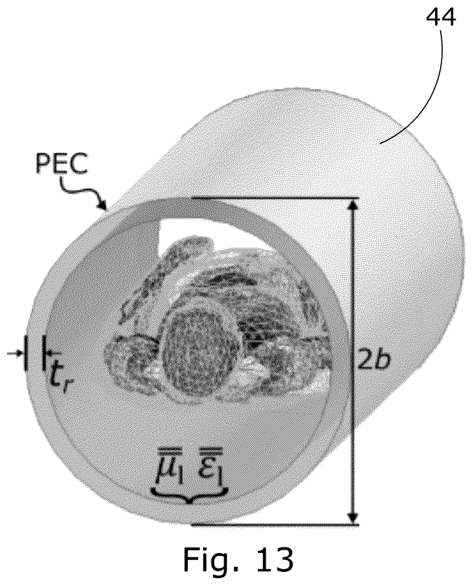

An objective of transmit (Tx) radio-frequency (RF) coil design for MRI is to produce homogeneous transverse RF magnetic fields efficiently for MR excitation of a targeted imaging region of interest (ROI). The excitation (B₁) fields must be generated without creating concentrated electric fields, and thus, local specific absorption rate (SAR) that exceeds the IEC safety limits. The transverse magnetic field for the 1st-order transverse electric (TE) mode of a circular waveguide are largely homogeneous, while producing electric fields that are also spread out more uniformly than conventional MRI coils, and therefore may produce lower local SAR for the same B₁. As outlined above, the design of a metamaterial liner is disclosed, which consists of concentric rings, separated longitudinally and placed on the inside of the MRI bore (FIG. 13). The derivation of the metamaterial properties and tuning for operation of the desired cylindrical waveguide mode at the Larmor frequency (4.7 T-200 MHz) demonstrates that such a metamaterial liner structure could effectively produce a B₁ field suitable for MRI. Thus, this work details the practical design of a whole-body metamaterial liner of finite length, comparing it to conventional MRI RF excitation coils using the metrics described below.

As the static magnetic field strength of MRI (B₀) increases the signal-to-noise received from a sample dominated resonator/coil increases proportionally, neglecting wavelength effects. Additionally, the frequency of operation ƒ₀ increases proportionally by the Larmor equation ƒ₀=γB₀, where γ is the gyromagnetic ratio of the nucleus (typically ¹H, γ=42.7 MHz/T). The field strength of 4.7 T is chosen in an example herein, since it is the first commonly available nominal field strength above the clinical 3 T where local SAR and homogeneity begins to severely constrain the application of many MR imaging methods in the trunk and internal organs (e.g., abdominal, thoracic, heart, lung). However, the methods and liner described herein can be applied to any field strength or bore size. The Larmor frequency greatly affects the key performance metrics of transmit coils for MRI. The most significant metrics are:
1. The transmission efficiency (η_{Tx}), which is a measure of the MR-relevant right circularly polarized magnetic field (B₁⁺) produced for a given input power (units of μT/\sqrt{kW}). The transmission efficiency is reported as the mean within a suitable region of interest (ROI).
2. The transmit homogeneity (σ_{Tx}), measured as the percent standard deviation (CoV) of the η_{Tx} within the ROI.
3. The specific absorption rate (SAR). At high B₀ MRI safety constraints are typically limited by the local 10 g average SAR (units of W/kg). The constraint imposed on the transmit field is expressed by reporting the SAR generated relative to the η_{Tx} ((W/kg)/μT²).

There are therefore three associated key challenges in RF Tx coil design at field strengths ≥3 T that motivate aspects of this work:
1. The ohmic losses in conductive tissue are roughly proportional to frequency squared: thus η_{Tx} ∝˜ω₀.
2. For B₀≥3 T (ƒ₀≥128 MHz) the wavelength (λ) inside the human body (with bulk relative permittivity ˜60ε₀) is short relative to the dimensions of the human body (<30 cm). The interference patterns thus increase the σ_{Tx}.
3. The interference patterns and higher power requirements to produce the same Tx field results in increased local 10 g SAR, which constrain the Tx field amplitude and duty cycle that may be safely used in MRI scanning.

In addition, for whole-body excitation the access to the center of the MRI scanner bore 34 must not be impeded. Except for dedicated localized Tx coils, the RF applicator is restricted to a minimal lining around the scanner bore's cylindrical surface.

Numerous approaches have been taken in RF coil design to address these challenges and a few popular volume coil designs have emerged: the TEM coil, the birdcage coil (BC), and transceive arrays using resonant elements such as microstrips or dipoles. The SAR with the TEM coil and BC is comparable, with some studies showing higher SAR for the BC and others showing higher SAR for the TEM. In general, if optimal designs are used to compare each Tx coil type, the η_{Tx} will be similar when they have similar dimensions and/or the same B₁ coverage area, as the losses will be dominated by losses in the body. Thus, at high fields the efficiency is constrained by the electric field associated with the desired B₁ field, which will be similar for all coil types.

Reduction in SAR can be achieved with transceive arrays, where improving the σ_{Tx}, while also reducing SAR, is still an area of ongoing research. There are many array element designs that attempt to improve metrics 1-3 without, or in addition to, high-density element shimming (i.e., beamforming) techniques. In the head at 7 T, the “tic-tac-toe” coil has been shown to have lower SAR, higher η_{Tx} and lower σ_{Tx} than a TEM coil with similar dimensions. The original travelling wave (TW) MRI has been modified to improve the efficiency while maintaining low SAR by the use of stepped impedance resonators to focus the fields, by using different exciting patch antenna or resonator designs, or by modification of the waveguide structure itself so that propagation is no longer dominated by the scanner bore (e.g., by using a parallel plate waveguide structure in reference). In addition, TW structures like the helical antenna have been shown to efficiently produce the desired transmit field distribution, while also allowing the currents to be distributed along the bore, and at high frequencies (>7 T), when operating in the fast-wave/axial mode regime, circularly polarized fields are naturally produced.

A theoretical description has been developed for a thin lining of metamaterial (MTM) that reduces the cutoff of hybrid transverse electric (HE) and hybrid transverse magnetic (EH) modes in circular metallic waveguide environments. A fully anisotropic exemplary MTM liner was produced using periodically reactively loaded and axially stacked rings, and was subsequently found to also improve Tx performance. Disclosed earlier herein, an accurate effective medium model was presented for the MTM liner and a method of design for a MTM lined MRI bore was introduced. The practical design and implementation of the MTM liner as an MRI coil is disclosed. The practical implementation first requires developing a network model that can predict the current pattern, input impedance and tuning of the MTM liner. Subsequently, the application for MRI is investigated by comparing the MTM liner to the conventional BC according to the Tx metrics described above.

The geometric description and network analysis of the exemplary MTM liner is presented herein, demonstrating three different reactive-loading scenarios resulting in a variety of useful propagation dispersion features, and engineered for the desired current distribution and resonance mode for MRI at 4.7 T (200 MHz). Additionally herein, full-wave simulations of the MTM liner are compared for the three cases to results from network analysis, demonstrating the dispersion characteristics and the expected transverse field distributions. Further herein, the operation of the full-scale MTM liner is evaluated by simulation of the MR relevant Tx performance metrics (ηTx, σTx and SAR) with a realistic human body model.

The exemplary MTM liner disclosed herein is a practical realization of the geometry shown in FIG. 13. A lining of anisotropic material with a thickness tᵣ, relative permittivity ε̅ₗ̅ and relative permeability μ̅ₗ̅ on the inside of a circular waveguide bounded by a perfect electric conductor (PEC) of radius b alters the propagation characteristics of its waveguide modes. Disclosed earlier herein, the dispersion of the effective material properties (assumed to be diagonal tensors ε̅ₗ̅=ε₀diag(εᵣ, εᵣ, ε₀), μ̅ₗ̅=μ₀diag(1, 1, μ_{z}), was derived for closely-spaced rings for EH₀₁ and each HEₙ₁ mode (n is the azimuthial mode order). These rings included periodically-arranged radial inductors between rings and ground (PEC), and azimuthally directed capacitors that can be tuned to achieve the desired mode and longitudinal propagation at the desired frequency. Furthermore, it was demonstrated how different propagation and mode-spacing characteristics were observed for εᵣ negative and near zero (ENNZ) with μ_{z}≅1-5, compared to μ_{z} negative and large (MNL), i.e., μ_{z}<−102, with εᵣ near zero but positive.

Herein, each constituent ring of the exemplary MTM liner is realized using the structure shown in FIG. 14A, consisting of 8 identical sections repeated azimuthally. ϕ indicates azimuthal angle and z axial direction. Differences of the structure from that of J. G. Pollock and A. K. Iyer, “Experimental Verification of Below-Cutoff Propagation in Miniaturized Circular Waveguides Using Anisotropic ENNZ Metamaterial Liners,” IEEE Transactions on Microwave Theory and Techniques, vol. 64, (4), pp. 1297-1305, 2016, which is hereby incorporated by reference in its entirety, include employing discrete longitudinal connections (reactances) that permit increased distance (corresponding to Δz shown in FIG. 14A) between rings and adds a degree of freedom for tuning the structure. An extension of the theory disclosed earlier herein is used for analysis. The section close-up shown in FIG. 14B (a single segment of a ring) includes components and dimensions that may be adjusted to tune the MTM liner:
Radially oriented (r-directed) capacitors (C^{r}) and inductors (L^{Mr}), with short connections between ground and the ring. Radial capacitor C^{r} is here shown implemented using overlapping surfaces with length lᵣ and width wᵣ.
Longitudinally oriented (z-directed) capacitors (C^{z}) and inductors (L^{Mz}), which connect adjacent rings together. Longitudinal capacitor C^{z} is here shown implemented as two capacitors each using overlapping surfaces of longitudinal length l_{z} and circumferential width w_{z}, and separated by thickness tₛ, and longitudinal inductor L^{Mz} also uses two inductors.
Azimuthally oriented (ϕ-directed) capacitors (C^{ϕ}). Azimuthally oriented capacitor C^{ϕ} is here shown implemented using overlapping surfaces with length l_{ϕ} and width w_{ϕ}.

A L^{Mϕ} is not represented explicitly in the diagram, since it would not be added to the large intrinsic inductance of the ring's conductive segments. Practically, the inductors may be lumped air-core inductors, while the capacitors may be, for example, either high quality-factor chip capacitors, or parallel-plate capacitors, as shown in FIG. 14B. The parallel plate capacitor implementation consists of a length (l_{ϕ,r,z,}) and width (w_{ϕ,r,z}) of overlapping conductor separated by the ring's structural dielectric substrate, with thickness tₛ and permittivity εₛ.

The full-wave simulations in this study were performed with the finite element full-wave simulator HFSS (Ansys, Canonsburg, PA). A simplified model is shown in FIG. 14C, which is expected to be a close approximation of the physical model in FIGS. 14A and 14B. The physical components C^{z}, L^{Mz}, C^{r}, L^{Mr}, and C^{ϕ} are replaced with lumped series reactances corresponding to the equivalent circuits of chip capacitors and air-core inductors (with inductance found from equations for single layer air core solenoid coils). For example, Z^{ϕ} replaces C^{ϕ}, Z^{z} replaces C^{z} and L^{z}, and Z^{r} replaces L^{Mr}. The network model developed herein below applies to both physical and simplified models. Full-wave simulation, however, does not provide a comprehensive or intuitive understanding of the operation of the MTM liner and requires substantial computing resources and time, thus precluding extensive exploration of the parameter space necessary for optimization. Therefore, a method of predicting the tuning frequency and input impedance from a few design parameters is a valuable tool to design the MTM liner and compare its behaviour to the BC, the TEM coil, or other MRI transmit coils. so an analytic (network) model, and method of analysis using the model is developed.

The propagation of travelling waves in an MTM lined waveguide is dominated by the MTM liner structure itself, which closely resembles a two-dimensional transmission line. Lumped element network models can accurately represent transmission lines (coplanar lines, coaxial lines, etc.). Thus, the network representation developed here mimics that for two-dimensional transmission-line matrix methods.

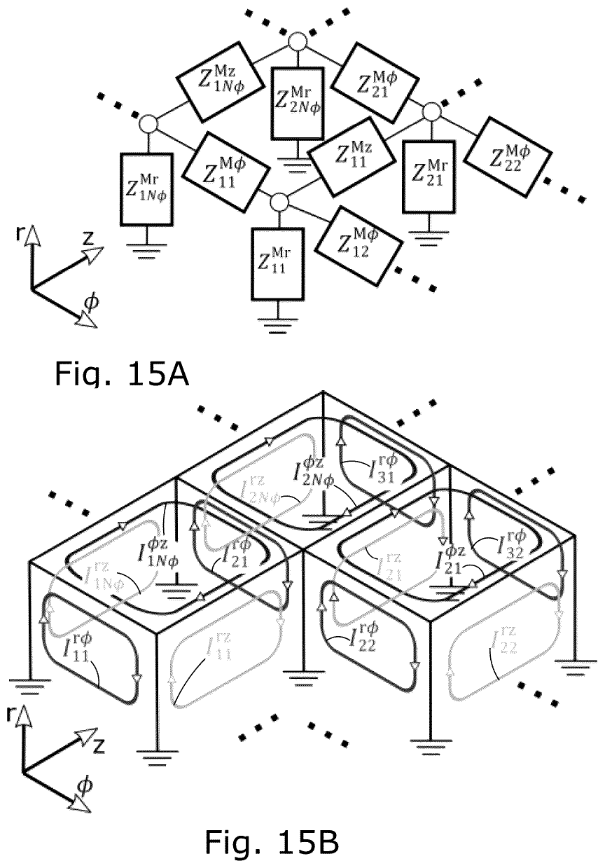

The model shown in FIG. 15A is a general lumped-circuit network representation of the interconnected ring structure of the MTM liner and includes the tuning elements shown in FIG. 14. To allow for different tuning components on different rings and different segments of the same rings, the subscripts n_{z} and n_{ϕ}, enumerating position axially and circumferentially respectively, are explicitly included. So, for example, Z₁₂ indicates an impedance associated with the first ring and second mesh on that ring. Thus, there are N_{ϕ} total meshes in the ϕ-(azimuthal) direction associated with each ring, with the N_{ϕ}-th mesh connected to the first. Similarly, the network model considers N_{z} total rings spaced in the z-(longitudinal) direction. The superscripts Mᵣ, M_{z} and M_{ϕ} indicate the direction of the impedance, i.e. in terms of what components it connects.

The network model of FIG. 15A shows only the lumped impedances of the network, consisting of LC models of chip or parallel plate capacitors, stray capacitances and air-core inductors in the implementation presented here. It does not show the intrinsic inductances of the MTM liner structure, which for this model are included as “geometric” inductances (L^{Gr,ϕ,z}) in series, so the total branch inductances (L^{r,ϕ,z}) are

L^{r,ϕ,z}=L^{Mr,ϕ,z}+L^{Gr,ϕ,z}. (C1a)

The mesh current paths of the ring structure are depicted in FIG. 15B, each of which is associated with an intrinsic self inductance that defines the values of L^{Gr,ϕ,z}, as well as having an associated mutual inductance with every other mesh. In the network model of the lumped impedances, there are N_{ϕ}×N_{z} of the ϕ−, , and r-directed impedances (Zₙ_{_{z}}ₙ_{_{ϕ}}^{ϕ} and Zₙ_{_{z}}ₙ_{_{ϕ}}^{r}), but N_{ϕ} ×(N_{z}−1) z-directed impedances (Zₙ_{_{z}}ₙ_{_{ϕ}}^{z}). The mesh currents and voltages are matrices expressed as

$$I = \begin{matrix}[ {I}_{1 ( 1 \to {N}_{ϕ} )}^{r ϕ} & {I}_{2 ( 1 \to {N}_{ϕ} )}^{r ϕ} & … & {{I}_{{N}_{z} ( 1 \to {N}_{ϕ} )}^{r ϕ} ]}^{†} , \\ [ {I}_{1 ( 1 \to {N}_{ϕ} )}^{ϕ z} & {I}_{2 ( 1 \to {N}_{ϕ} )}^{ϕ z} & … & {{I}_{( {N}_{z} - 1 ) ( 1 \to {N}_{ϕ} )}^{ϕ z} ]}^{†} , \\ [ {I}_{1 ( 1 \to {N}_{ϕ} )}^{rz} & {I}_{2 ( 1 \to {N}_{ϕ} )}^{rz} & … & {{I}_{( {N}_{z} - 1 ) ( 1 \to {N}_{ϕ} )}^{rz} ]}^{†}\end{matrix} \qquad ( C2a )$$

$$V = \begin{matrix}[ {V}_{1 ( 1 \to {N}_{ϕ} )}^{r ϕ} & {V}_{2 ( 1 \to {N}_{ϕ} )}^{r ϕ} & … & {{V}_{{N}_{z} ( 1 \to {N}_{ϕ} )}^{r ϕ} ]}^{†} , \\ [ {V}_{1 ( 1 \to {N}_{ϕ} )}^{ϕ z} & {V}_{2 ( 1 \to {N}_{ϕ} )}^{ϕ z} & … & {{V}_{( {N}_{z} - 1 ) ( 1 \to {N}_{ϕ} )}^{ϕ z} ]}^{†} , \\ [ {V}_{1 ( 1 \to {N}_{ϕ} )}^{rz} & {V}_{2 ( 1 \to {N}_{ϕ} )}^{rz} & … & {{V}_{( {N}_{z} - 1 ) ( 1 \to {N}_{ϕ} )}^{rz} ]}^{†}\end{matrix} \qquad ( C2b )$$

where † indicates transpose, and the superscript indicates whether the currents or voltages correspond to the rϕ, ϕz, or rz oriented meshes in FIG. 15. Subscripts have substantially the same meaning as with the impedances of FIG. 15A, except that these current loops extend over multiple junctions and are labeled based on a single corner, oriented consistently between the different loops of common orientation. The general network equation that relates the matrices of currents and voltages is simply

I=(Z^{G}+Z^{M})⁻¹V=Z⁻¹V. (C3)

The impedance matrix Z accounts for the lumped impedances in FIG. 15A, as well as the geometrically dependent inductances of the current meshes in FIG. 15B. To account for nearest neighbor mutual inductance between meshes of different rings, such as the meshes associated with I₁₁^{rϕ} and I₂₁^{rϕ} in FIG. 15B, only three key mutual inductance terms (M^{rϕrϕ},M^{ϕzϕz},M^{rϕϕz}) were included to account for the magnetic flux between the nearest-neighbor mesh currents. See herein below for details on how the elements of the impedance matrix (Z^{r,ϕ,z}) and mutual inductance terms are assembled. Commercial circuit simulators can solve the network model, but the mutual impedance terms are not easily included. Implementation is far less cumbersome using the concise matrix method implemented here in MATLAB. Results were validated by comparison (not included) to Ansys Circuit.

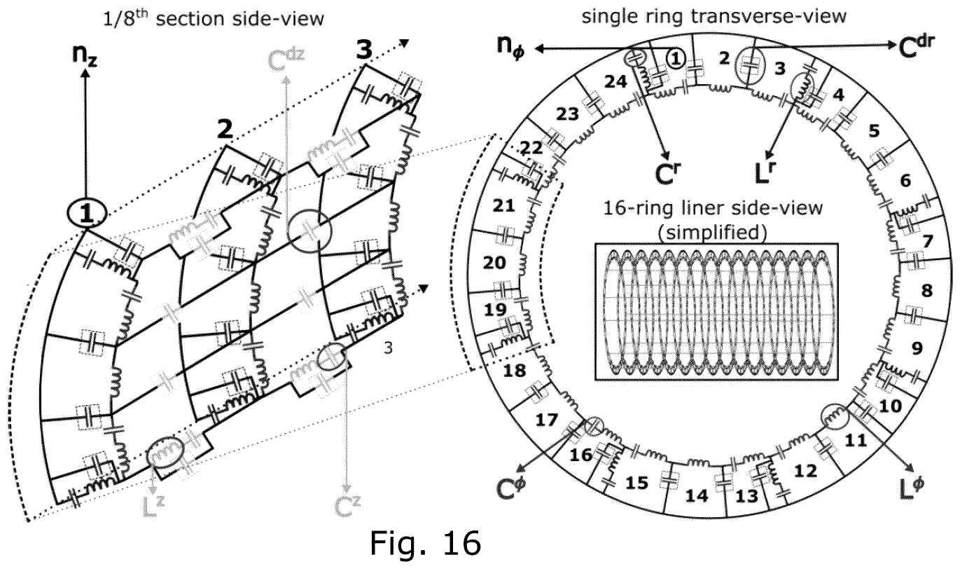

The case of two physically-separate C^{ϕ} tuning capacitances between each radial impedance, here combined radial capacitance and inductance C^{r} and L^{r}, matches the case shown in FIG. 16, which best represents the distributed capacitance obtained by the overlapping strips in FIGS. 14A and 14B. Also, the distributed inductance and parasitic capacitances (C^{dr}, C^{dϕ} and C^{dz}) are represented more faithfully by including multiple azimuthal sub-segments for each radial element. In this example, three mesh sub-segments are included for every radial element, so that the total number extending around the circumference is N_{ϕ}=24 in FIG. 16. The sub-segments are numbered around the circumference with numbers n₉₉ , and axially adjacent elements with numbers n_{z}. All elements of Zₙ_{_{z}}ₙ_{_{ϕ}}^{ϕ,r,z} are represented by series LC circuits in parallel with stray distributed capacitances between adjacent ring conductors (C^{zd}), between the ring conductor and ground (C^{rd}) and between azimuthal segments (C^{ϕd}). Additionally, for a symmetrically constructed MTM liner all mesh tuning elements and geometric parameters will be equal for each n_{z} and n_{ϕ}. To exclude an n_{z}n₉₉ lumped inductor or capacitor we set jωLₙ_{_{z}}ₙ_{_{ϕ}}^{ϕ,r,z}=1/jωCₙ_{_{z}}ₙ_{_{ϕ}}^{ϕ,r,z}=0. Therefore, the elements of Z^{r,ϕ,z} are:

$${Z}_{{n}_{z} {n}_{ϕ}}^{r} = \{ \begin{matrix}\frac{( j ω {L}^{r} + 1 / j ω {C}^{r} ) / j ω {C}^{rd}}{j ω {L}^{r} + 1 / j ω {C}^{r} + 1 / j ω {C}^{rd}} , & {n}_{ϕ} = 3 , 6 , 9 , … {N}_{ϕ} \\ 1 / j ω {C}^{rd} , & otherwise\end{matrix} \qquad ( C4a )$$

$${Z}_{{n}_{z} {n}_{ϕ}}^{ϕ} = \{ \begin{matrix}\frac{( j ω {L}^{ϕ} + 1 / j ω {C}^{ϕ} ) / j ω {C}^{ϕ d}}{j ω {L}^{ϕ} + 1 / j ω {C}^{ϕ} + 1 / j ω {C}^{ϕ d}} , & {n}_{ϕ} = 1 , 3 , 4 , 6 … {N}_{ϕ} \\ {L}^{ϕ} , & otherwise\end{matrix} \qquad ( C4b )$$

$${Z}_{{n}_{z} {n}_{ϕ}}^{z} = \{ \begin{matrix}\frac{( j ω {L}^{z} + 1 / j ω {C}^{z} ) / j ω {C}^{zd}}{j ω {L}^{z} + 1 / j ω {C}^{z} + 1 / j ω {C}^{zd}} , & {n}_{ϕ} = 3 , 6 , 9 , … {N}_{ϕ} \\ 1 / j ω {C}^{zd} , & otherwise\end{matrix} \qquad ( C4c )$$

The MTM liner is a complex EM structure with many tuning and geometry-dependent parameters. Thus, this section explores the impact of each variable on the overall behavior of the structure. Unlike with other MRI resonators (loop coils, BC, etc.) the MTM structure must be analyzed by considering the fields and current/voltage distributions as longitudinally propagating waveguide modes with complex propagation constants defined as γₙ(ω)=αₙ(ω)+jβₙ(ω), where the propagation constant is dependent on the mode order n. The βₙ(ω) represents propagation and will be reported here in relation to the free-space wavenumber k₀=ω/c. The αₙ(ω) is associated with attenuation and decaying or evanescent modes. The propagation constant is used to determine the tuning values for a given length of MTM liner. Specifically, for an efficient excitation in the middle of the MTM liner a length of βₙ(ω)ΔzN/hd z/2π=m_{z}˜1/2 is required, where m_{z} is defined here as the longitudinal resonance mode order: m_{z} ={0,1/2,1,3/2 . . . }.

Unlike conventional MRI resonators (coil arrays, BCs or TEM coils) the MTM liner produces longitudinal propagating modes by facilitating the operation of the scanner bore as a waveguide. Thus, the MTM liner must be analyzed and understood in terms of its propagation characteristics, along with its frequency-dependent input impedance and current and field distributions. The complex propagation constant γₙ (ω) and the radial distribution of the fields defined by H_{r,ϕ,z}(r) and E_{r,ϕ,z}(r) have been derived in previous work. The EM fields of the different modes of the structure shown in FIG. 13 are represented in a simplified form as

H=e^{-γn(ω)z}(Hᵣ(r) sin(nϕ)\hat{r}+H_{ϕ}(r) cos(nϕ)\hat{ϕ}+H_{z}(r) sin(nϕ)\hat{z})

E=e^{-γn(ω)z}(Eᵣ(r) cos(nϕ)\hat{r}+E_{ϕ}(r) sin(nϕ)\hat{ϕ}+E_{z}(r) cos(nϕ)\hat{z}). (C5)

They are dependent on the azimuthal mode order (n), angular frequency (ω), ε̅ₗ̅ and μ̅ₗ̅. For each value of n there are two classes of solutions, the HEₙ₁ and EHₙ₁ modes, whose cutoffs are most dependent on {ε_{ϕ}, μ_{z}} and {ε_{z}, μ_{ϕ}}, respectively. Only the HEₙ₁ modes (the second indices in HEₙ₁ indicates radial mode order) and EH₀₁ mode are investigated, since the ring structure under investigation supports primarily azimuthal currents that affect μ_{z} and ε_{ϕ}. To control the fields and prevent mode mixing, the MTM liner is designed such that the propagation of the EHₙ₁ and HEₙ₁ modes do not occur over the same frequency range.

The focus may be on the real part of the propagation constant, βₙ, which determines the longitudinal resonance modes over the finite length of the MTM liner. For an MTM resonator to work efficiently it will also require αₙ(ω)<<β(ω), so that over the length of the imaging region the decay is small.

To relate the results of the network model to the propagation constant of the MTM liner, the currents on the outer conductor (PEC boundary) of the bore are modelled as

$$I ( b ) = {\sum }_{n} {e}^{- {γ}_{n} ( ω ) z} ( {A}_{n} cos ( n ϕ ) \hat{z} + {B}_{n} {I}_{ϕ} sin ( n ϕ ) \hat{ϕ} ) . \qquad ( C6 )$$

I(b) is continuous in the real structure of FIG. 13, but for the practical network model of the MTM liner in FIG. 16, the discrete form of (C6) is used to represent the currents in (C2a)

$${I}^{r ϕ , ϕ z} = {\sum }_{n} {A}_{n}^{r ϕ , ϕ z} {e}^{- {α}_{n} ( ω ) {n}_{z} Δ z} {e}^{- j {β}_{n} ( f ) {n}_{z} Δ z} sin ( \frac{2 π {n}_{ϕ} n}{{N}_{ϕ}} ) \qquad ( C7 )$$

$${I}^{rz} = {\sum }_{n} {A}_{n}^{rz} {e}^{- {α}_{n} ( ω ) {n}_{z} Δ z} {e}^{- j {β}_{n} ( f ) {n}_{z} Δ z} cos ( \frac{2 π {n}_{ϕ} n}{{N}_{ϕ}} ) .$$

Therefore, βₙ (ω) is found by applying the discrete Fourier transform in the longitudinal direction (z=n_{z}Δz) to I^{rϕ}. Under the assumption that an αₙ≅0 this results in

$${ℱ}_{Z} ( {I}^{r ϕ} ) = \sum {A}_{n}^{r ϕ} cos ( \frac{2 π {n}_{ϕ} n ϕ}{{N}_{ϕ}} ) δ ( ξ - \frac{{β}_{n} ( ω )}{2 π} ) . \qquad ( C8 )$$

where ξ is the spatial frequency in the Fourier transform domain. The modes are found by identifying the peaks in the resulting spectrum, i.e., βₙ (ω)'2πξ. There may be two peaks at each ω if two modes of different dispersions are supported simultaneously (i.e., degenerate). For αₙ≠0 a Lorentzian distribution is obtained instead with the same central peaks. Since modes of different order or class (EH or HE) are orthogonal, individual HEₙ₁ modes can be exclusively excited by inducing a voltage distribution on the first (n_{z}=1) ring proportional to

$${V}_{1 {n}_{ϕ}}^{r ϕ} = cos ( \frac{2 π {n}_{ϕ} n}{{N}_{ϕ}} ) {V}_{1 {n}_{ϕ}}^{ϕ z} = - cos ( \frac{2 π ( {n}_{ϕ} - 1 ) n}{{N}_{ϕ}} ) . \qquad ( C9 )$$

Discrete values of

$$\frac{{β}_{n} ( ω )}{2 π} = \{ 1 , 2 , … {N}_{z} / 2 \} / {N}_{z}$$

are obtained from (C8) using this method. To avoid staircasing, the βₙ (ω) is plotted only at the frequencies corresponding to these points.

In summary, to predict the propagation constant from the network model of the MTM liner, for each n and frequency point, the excitation of (C9) is applied, the currents are obtained from (C3), then the Fourier transform in (C8) is applied and the βₙ (ω) is obtained from the spectral peaks.

The network model is applied to the MTM liner model shown in FIG. 14C and compared to HFSS simulation. The dimensions of the structure are wₛ=12.5 mm, tᵣ=10 mm, tₛ=1.56 mm, εₛ=3.4, b=275 mm, Δz=62.5 mm. The azimuthal length of each mesh sub-segment is 2πα/N_{ϕ}=6.94 cm, which s<<λ=1.5 m at 200 MHz and satisfies the assumption of electrically small components for lumped element analysis. The eigenmode solver in HFSS was used with master-slave boundaries on the opposite cylindrical faces of the MTM liner unit cell shown in FIG. 14C. The iterative solver used a mesh of 155815 tetrahedra (first-order basis functions with (979410)² matrix) to a convergence of 0.03% frequency error, requiring 20.2 GB RAM with two 8-core Xeon processors and 15.4 minutes solver time for each specified phase point (every 6° up to 180°) between master-slave boundaries. For comparison, the network model requires the inversion of Z for each frequency point, which is a 4,608×4,608 sparse matrix and requires much fewer computing resources than the HFSS simulation (can be performed on a desktop computer with <2 GB RAM).

It was disclosed earlier herein that in the case that the MTM structure includes radial inductors that are close to resonant with the stray capacitance between ring conductors segments and ground the cutoff of HEₙ₁ modes will occur below the EH₀₁ mode and in the mode's backward-propagating region the reactance of the azimuthal segments will be capacitive and effective ε_{ϕ} will be ENNZ. Additionally, close to the HE₀₁ mode, where the series azimuthal reactance is near-zero, the cutoff of forward propagating HEₙ₁ modes will occur at higher frequencies when the azimuthal reactance are inductive for radial capacitors (high-pass (HP)) or lower frequencies when the azimuthal reactance are capacitive for radial inductors (low-pass (LP)) and that the effective μ_{z} will be MNL (ε_{ϕ} near zero at cutoff and positive in propagating region). These, three different cases are considered in this study, as indicated below:
ENNZ, C^{ϕ}=1 pF, L^{rM}=107.5 nH,
HP-MNL, C^{ϕ}=15.8 pF, C^{r}=20 pF,
LP-MNL, C^{ϕ}=10 pF, L^{rM}32 30 nH.

The geometric parameters were adjusted to achieve approximately the same cutoff values, mode spacing and dispersion slope with the network model as in the full-wave simulation, for all three cases, as described in the supplementary materials:
L^{rG}=2.5 nH, L^{zG}=19 nH, L^{ϕG}=31 nH, C^{ϕd}=0.5 pF, C^{rd}=3.3 pF, C^{zd}=0.5 pF, M^{ϕzϕz}=M^{rϕϕz}=7.43 nH, M^{rϕrϕ}=0.66 nH.

Additionally, variation of the longitudinal tuning parameters C^{z} and L^{z}, as described in the supplementary materials, was performed to determine the values that result in greatest slope in the dispersion diagrams (separation of longitudinal resonance modes, m_{z}) and prevent overlap of propagation of the HEₙ₁ modes at the Larmor frequency (4.7 T-200 MHz), for the three cases:
ENNZ, C^{z}=6 pF, L^{z}=0 nH,
HP-MNL, C^{z}=∞pF, L^{z}=0 nH,
LP-MNL, C^{z}=∞pF, L^{z}=30 nH.

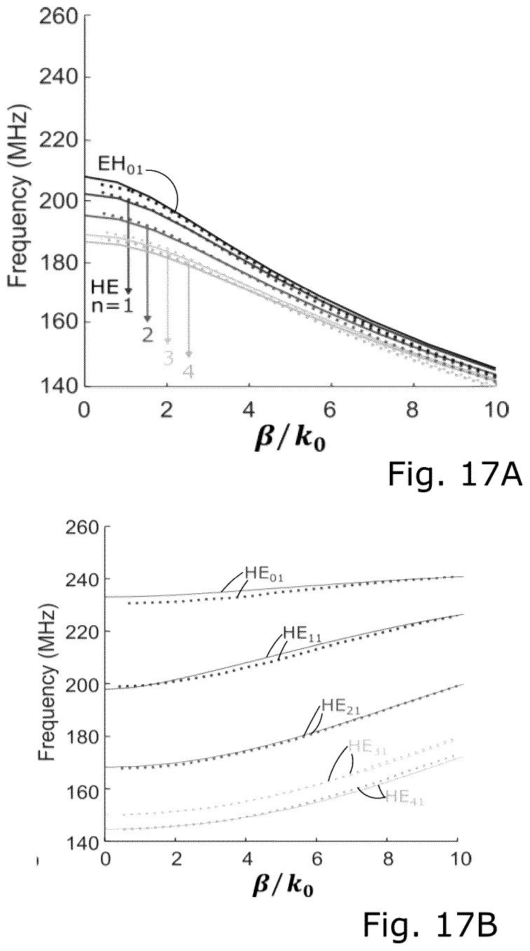

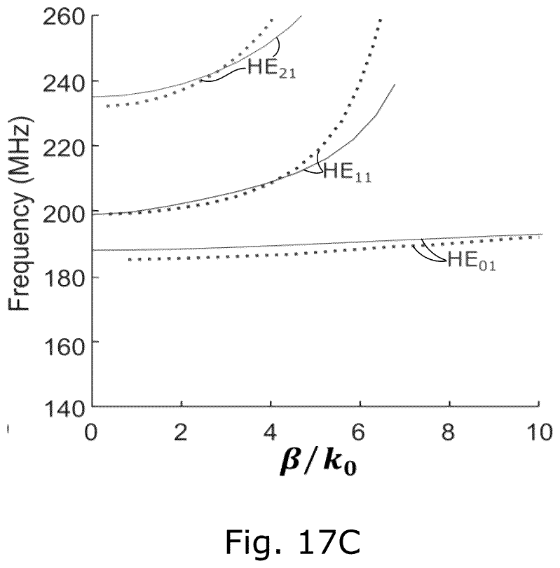

FIG. 17 shows the resulting β/k₀ dispersion diagrams for the MTM liner predicted using the network model, compared to those predicted using the HFSS eigenmode solver, with geometric and longitudinal tuning impedance parameters manually tuned to obtain the best match. The full-wave simulation is expected to be closer to the ground-truth because it accounts for mutual impedance between all meshes, the actual propagation contribution from the scanner bore RF shield, as well as allowing more general distributions of current and voltage across the MTM liner structure. However, the network model is abundantly accurate (within a few percent), so it is valuable in practice for design optimization and to provide working estimates of the tuning values. The model has the greatest discrepancy with the HP-MNL case since it is the most dispersive and has a stronger dependence on the longitudinal impedance.

It was disclosed earlier herein that the low Q-factor (<200) of large radial inductances will result in significant loss of transmit efficiency. For MRI excitation, low losses are required and only a single HE₁₁ longitudinal m_{z}=1/2 resonance must be excited. Only the MNL highpass case with direct longitudinal connections is free of other HEₙ₁ modes at 200 MHz and avoids the use of lossy inductors. Thus, the MNL high-pass case is best for the practical application envisioned here and considered for the rest of this study.

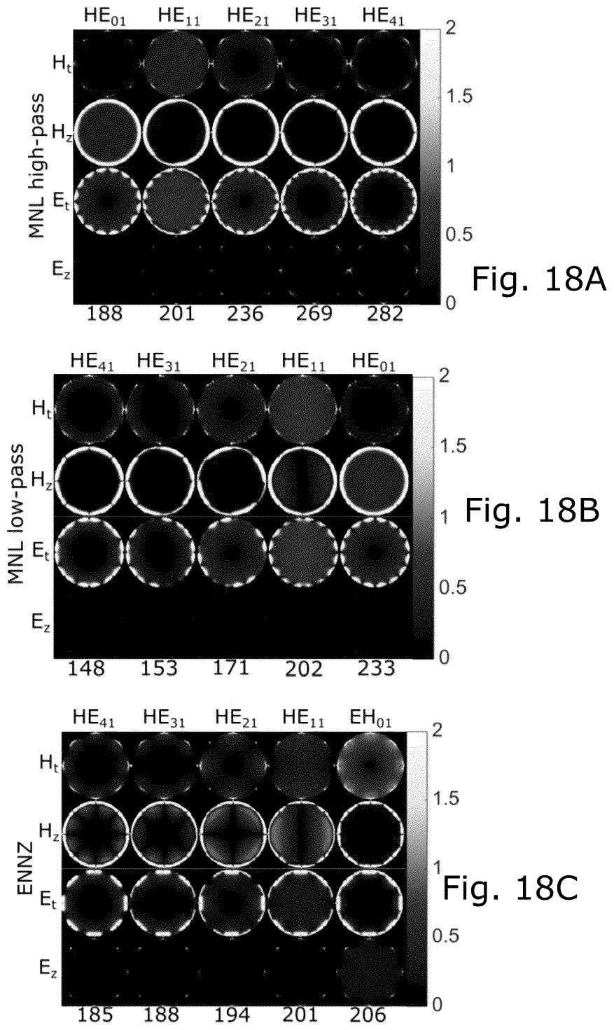

The longitudinal (E_{z}, H_{z}) and transverse (Eₜ, Hₜ) components of the simulated electric and magnetic field magnitudes in a central transverse plane of the eigenmode unit cell simulation (FIG. 14C) for the three cases are shown in FIG. 18. The HE₁₁ mode is the only one that provides the homogeneous transverse magnetic field desired for MRI excitation. For each HEₙ₁ mode the field distribution for the three cases is similar, with minor variations due to differences in the induced current distributions. The discrete structure of the MTM liner approximates the continuous structure of the ideal MTM liner depicted in FIG. 13, which results in a staircase or sampled approximation of the purely azimuthal sinusoidal distribution, resulting in regions of periodic nodes and peaks in the field magnitudes, especially near the circumference. The method of excitation and the boundaries at the ends of the liner influence the imposed current distribution; therefore, to take these effects into consideration it is necessary to evaluate the MTM liner as a resonator with finite length, as in the next section.

The 4.7 T scanner intended for experimental verification (Varian, University of Alberta, Peter S. Allen Research Center) produces image encoding magnetic field gradients with 4% linearity variation in a sphere with diameter of 25 cm, and so the RF resonator length is designed to approximately match this effective longitudinal coverage (N_{z}=16, 95 cm length). In our prior publication comparing the efficiency of a BC and MTM liner without longitudinal connections it was observed that for the same longitudinal coverage the MTM liner must be approximately double the length of the BC.

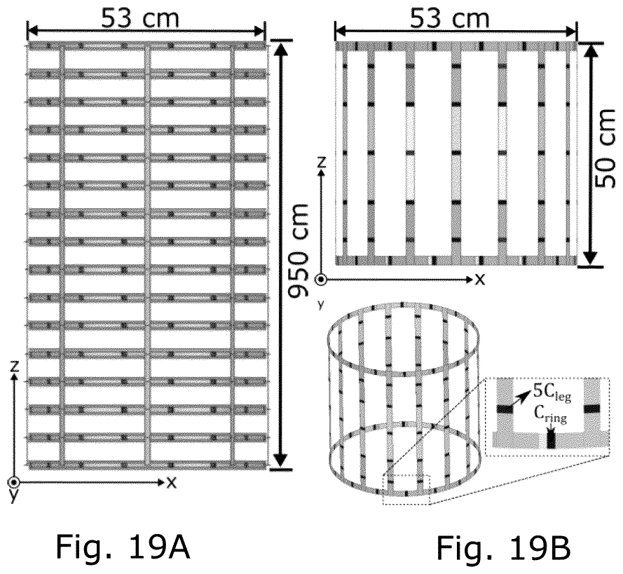

A full-scale MTM liner model is shown in FIG. 19A, and a BC used for comparison with comparable longitudinal coverage is shown in FIG. 19B. While an MTM liner would typically be used without a birdcage coil, a BC is also an example of an antenna that could be used in combination with an MTM liner, if desired. Other antennas could also be used. Generally, the MTM would be expected to be excited directly, e.g. using a cable as described above, or used with an antenna or multiple antennas of smaller axial length than a birdcage coil, to increase the room in the bore for the patient. The mention of an “antenna” in the singular in the claims should not be interpreted to exclude the presence of additional antenna(s). A single antenna may also have multiple independent connections. The values for the C^{ϕ} and C^{r} used in this model are those of the HP-MNL case of the MTM liner. The C_{ring} and C_{leg} capacitor values for the hybrid BC (16.3 pF and 7.2 pF, respectively) were derived by algebraic methods considering the simulated impedance matrix of the meshes of the BC. A 1.4 m long conductive shield with 55 cm diameter encompasses both designs in simulation to account for the outer conductor (RF shield) of the bore. Both the MTM liner and BC coil are fed by lumped ports placed in series with capacitors on the end-rings.

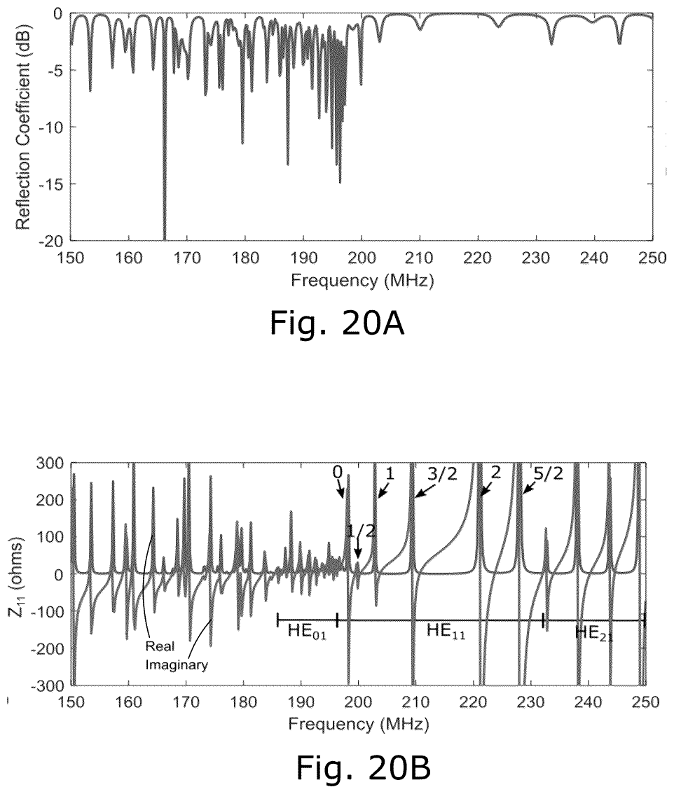

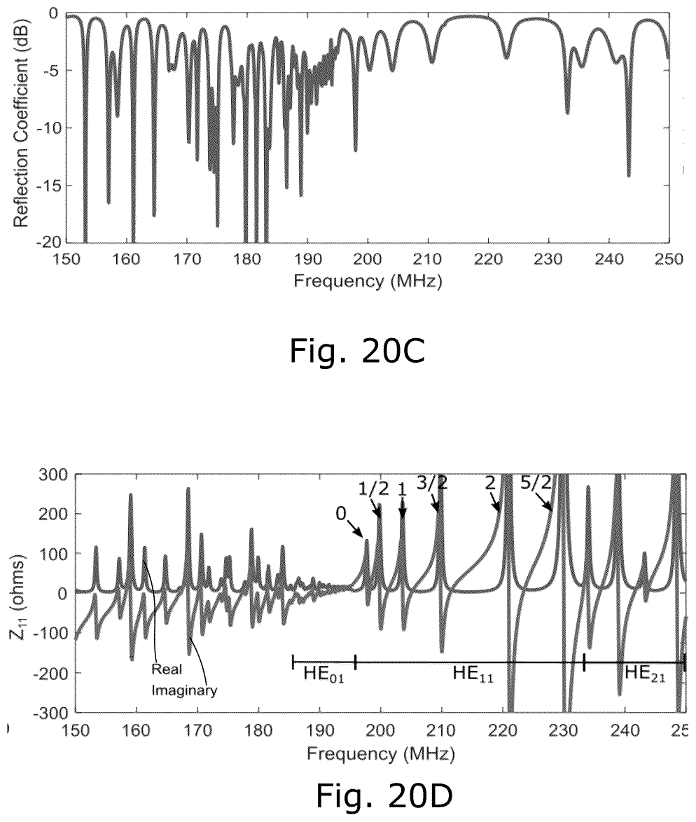

The reflection coefficient simulated using the network model in FIG. 16 and by full wave simulation of the structure shown in FIG. 19A are provided in FIG. 20A and FIG. 20C, respectively. In addition, the input impedance (real and imaginary) is shown in FIG. 20B and FIG. 20D. Since this is a truncated structure, standing waves are formed leading to resonance peaks in the reflection coefficient and input impedance associated with the longitudinal resonance modes (m_{z}). Note that such resonances could not exist in the bore alone because it is below cutoff, and are, in fact, enabled by the MTM liner.

Below and near the cutoff of the HE₀₁ mode, interfering resonances are observed due to propagating EHₙ₁ modes, which involve the longitudinal inductive connections and the capacitances of the azimuthal sections. The propagation of these modes terminates just below the cutoff of the HE₀₁ mode. The first few m_{z} longitudinal standing wave modes for the HE₁₁ mode are indicated by arrows in FIG. 20B and FIG. 20D. Although the frequencies and input impedance for the full-wave simulation and network model do not match exactly, the trend and approximate spacing between resonances have a similar level of agreement to analytical modeling vs. measurement or simulation of BC modes.

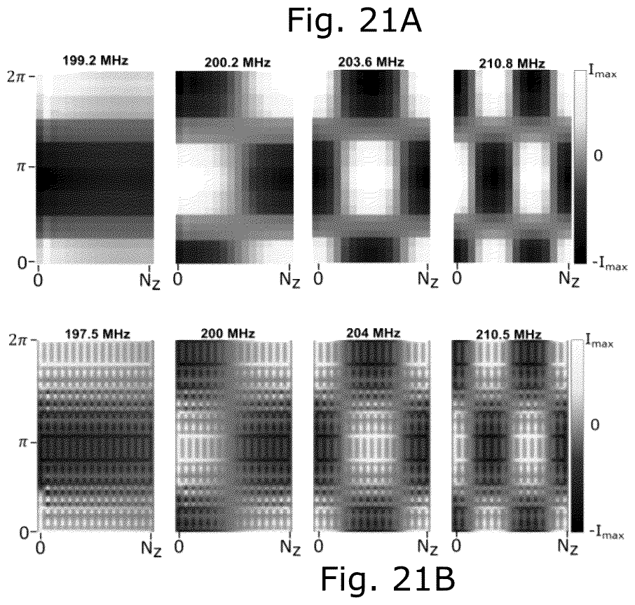

The azimuthally-directed current distributions for the first four resonances of the HE₁₁ mode simulated from the network model are compared to those on the cylindrical RF shield from the full-wave model in FIG. 21A and FIG. 21B, respectively. The currents on the shield mirror those on the rings, with nulls observed between the longitudinal spacings of the ring segments. Peaks in the full-wave simulated current distribution occur at the locations of the radial elements and longitudinal wires. The network model only represents the currents on the conducting wires of the MTM structure and does not fully reproduce all spatial EM aspects and the distributed impedances of the structure; these include, for example, mutual impedances between non-adjacent rings, the EM fields in the central free-space volume of the bore, and interaction of fringe fields at the terminating end rings with the MR bore. This may explain why the frequency spacing between the zeroth resonance and 1st m_{z}=1/2 resonance is smaller in the network model than in the simulation (FIG. 20 and FIG. 21).

The network model nevertheless captures the essential characteristics of the novel MTM liner and thus is a useful tool for design optimization and comparison. Calculation of the EM fields and MR-relevant metrics in presence of the complex geometry of the human body, however, requires numerical full-wave solvers.

As discussed above herein, the η_{Tx}, σ_{Tx} and efficiency normalized local 10 g SAR average are the three essential metrics for comparison of transmit coils for MRI. Comparisons of the metrics may be made with realistic loading from imaging subjects, and therefore the VHP female human body model 2.0 with standard human tissue EM values was used as the numerical MRI phantom. The body model was scaled to 92.5% of the original in the transverse direction to fit within the available diameter (matching that of the intended 4.7 T scanner). To include realistic losses, the conductive elements of the BC and MTM liner, as well as RF shield, were modelled as copper sheets (conductivity σ=5.8×107 S/m) with a surface roughness of 1 μm. Tuning was performed to achieve an isolation between quadrature ports of 25 dB or better with both the MTM liner and BC designs by adjusting the reactance of lumped ports on the diametrically opposite sides of the excitation ports. The input power for excitation was adjusted to deliver 500 W into each port with 90° phase difference.

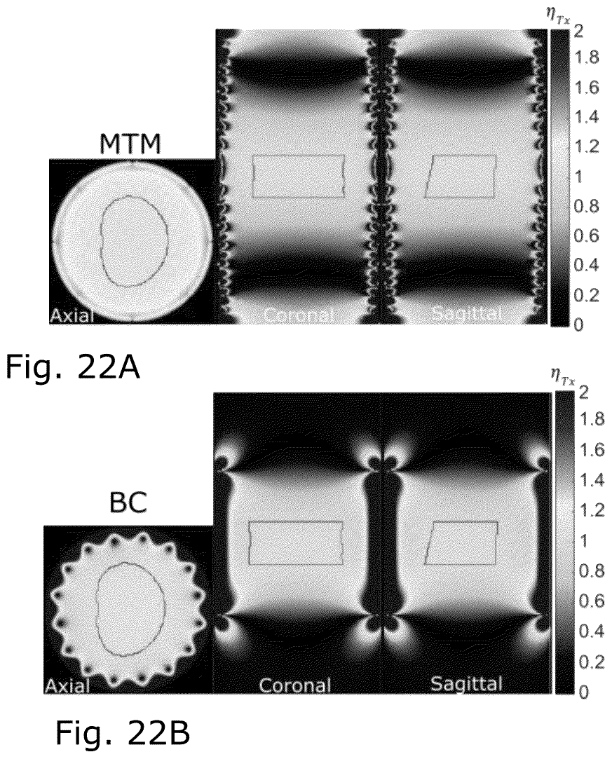

The results for the simulated B₁⁺ in the bore when it is empty are shown in FIG. 22. Both resonators have similar mean transmit efficiencies (within 3%) inside the outlined FOV (which covers a central section of the human body ±7.5 cm from the center longitudinally), as listed in Table 1. Additionally, the σ_{Tx} is very small (<2.5%) for both designs within the outlined FOV, and they both have similar longitudinal coverage. near the current-carrying rungs the BC has regions of inhomogeneity.

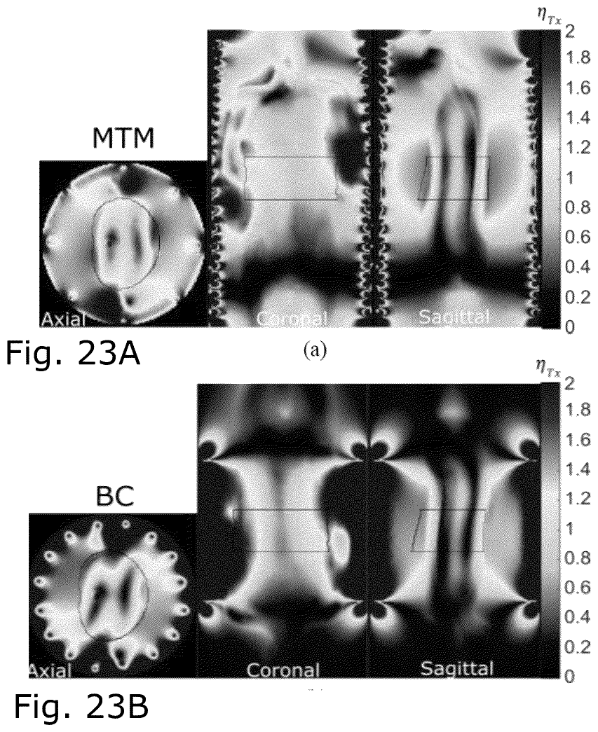

Interestingly, the transverse distribution of the simulated B₁⁺ within the human body model, shown in FIG. 23, is similar, showing an interference pattern with two nodes in the anterior posterior direction due to the dominant wavelength effects in the body. However, the MTM liner shows regions of much greater excitation near the head, which is near the excited end ring, that are not present in the empty bore simulation (FIG. 22). This is due to two factors: by acting as a waveguide there is a longitudinal reflection of the excited wave at the body boundary; and there is a mixing of the nearby 0^{th} HE₁₁ longitudinal resonance due to the reduced Q in presence of the body (bandwidth of the modes overlap). This should not negatively affect the performance in MRI excitation. In fact, a slightly larger η_{Tx} and lower σ_{Tx} is obtained for the MTM liner vs. BC with the body included. The overall large σ_{Tx} in the presence of the body is unavoidable with a single circularly polarized excitation but may be improved with additional excitation ports (maintaining the same CP excitation) or by using “beamforming” methods such as RF shimming, which may be implemented by placing input ports at different locations or combining the liner with resonators that have current distributions orthogonal to those of the MTM liner.

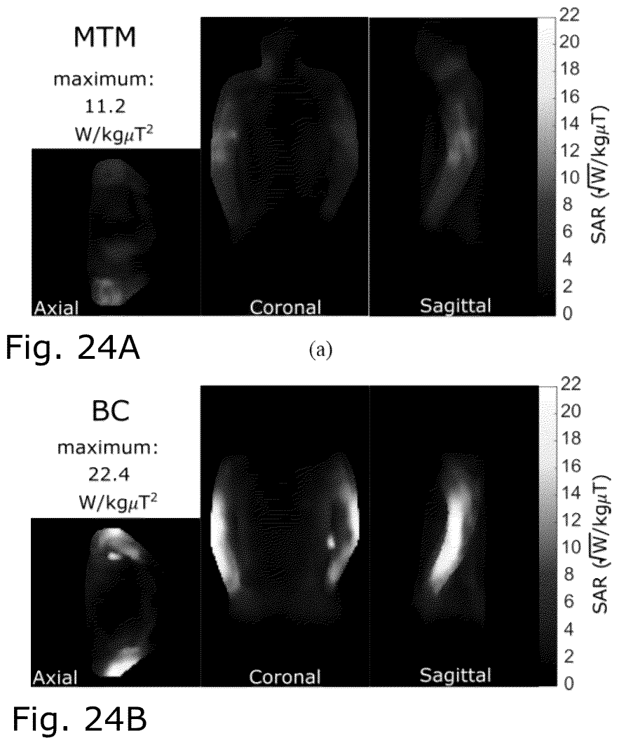

A difference between the performance of the BC and MTM liner for MRI is the local 10 g SAR in FIG. 24. To produce the same mean B₁⁺ the 10 g SAR maximum is reduced by a factor of 2, which is a remarkable advantage of the MTM liner. This is due to the distributed currents on the MTM rings, as opposed to the currents concentrated on two end rings and longitudinal rungs of the BC, resulting in more homogeneously distributed E-fields. Thus, in the MTM the SAR is redistributed towards the neck and legs and reduced at the periphery of the torso and arms. In high (>1.5 T) and ultra-high (>4.7 T) field MRI the application of many rapid imaging sequences is greatly limited by local SAR constraints. Thus, the reduced SAR of the MTM liner design improves temporal imaging efficiency (e.g., faster image acquisition).

A robust methodology for designing and evaluating the MTM liner as a whole-body transmit MRI coil is disclosed. This follows the introduction of the theoretical underpinnings of the MTM liner as was disclosed earlier herein. An example of a key benefit of the MTM liner is a 50% lower local 10 g averaged SAR, with homogeneity and transmit efficiency like the conventional birdcage coil. Therefore, the proposed MTM liner is a direct replacement for the birdcage in cases where local SAR is a constraint in faster scanning. The methods can be implemented equally well at the common clinical field strengths of 1.5 T and 3 T.

An impedance matrix may be derived as follows: The 3(N_{ϕ}N_{z})×3(N_{ϕ}N_{z}) impedance mesh matrix, Z, incorporates the Z^{r,ϕ,z} impedance matrices, and is subdivided into six (N_{ϕ}N_{z})×(N_{ϕ}N_{z}) blocks as:

$$Z = [ \begin{matrix}{Z}^{r ϕ} & {Z}^{ϕ zr ϕ} & {Z}^{rzr ϕ} \\ {Z}^{ϕ zr ϕ′} & {Z}^{ϕ z} & {Z}^{rz ϕ z} \\ {Z}^{rzr ϕ′} & {Z}^{rz ϕ z ′} & {Z}^{rz}\end{matrix} ] . \qquad ( C10 )$$

Z^{rϕ,ϕz,rz} describes interactions between the rϕ-rϕ,ϕz-ϕz,rz-rz meshes as:

$${Z}^{r ϕ , ϕ z , rz} = [ \begin{matrix}{Z}_{1}^{Rr ϕ , ϕ z , rz} & & - {Z}_{1}^{Cr ϕ , ϕ z , rz} & 0 & - {Z}_{{N}_{z}}^{Cr ϕ , ϕ z , rz} \\ & & & & 0 \\ - {Z}_{1}^{Cr ϕ , ϕ z , rz} & & ⋱ & & - {Z}_{{N}_{z} - 1}^{Cr ϕ , ϕ z , rz} \\ 0 & & & & \\ - {Z}_{{N}_{z}}^{Cr ϕ , ϕ z , rz} & 0 & - {Z}_{{N}_{z} - 1}^{Cr ϕ , ϕ z , rz} & & {Z}_{{N}_{z}}^{Rr ϕ , ϕ z , rz}\end{matrix} ] , \qquad ( C11 )$$

where the N_{ϕ}×N_{ϕ}Z_{nz}^{Rrϕ,ϕz,rz} “ring” matrices define the interaction within each ring given by:

$${Z}_{{n}_{z}}^{Rr ϕ} ( {m}_{ϕ} , {n}_{ϕ} ) = \{ \begin{matrix}{Z}_{{n}_{z} {m}_{ϕ}}^{r} + {Z}_{{n}_{z} {m}_{ϕ}}^{ϕ} + {Z}_{{n}_{z} ( {m}_{ϕ} + 1 )}^{r} , & {m}_{ϕ} = {n}_{ϕ} \\ - {Z}_{{n}_{z} \{ {n}_{ϕ} , {m}_{ϕ} \}}^{r} , & {m}_{ϕ} = \{ {n}_{ϕ} + 1 , {n}_{ϕ} - 1 \} \\ 0 , & otherwise\end{matrix} \qquad ( C12 )$$

$${Z}_{{n}_{z}}^{R ϕ z} ( {m}_{ϕ} , {n}_{ϕ} ) = \{ \begin{matrix}{Z}_{{n}_{z} {m}_{ϕ}}^{z} + {Z}_{{n}_{z} {m}_{ϕ}}^{ϕ} + {Z}_{{n}_{z} {m}_{ϕ} - 1}^{z} , {Z}_{{n}_{z} - 1 ) {m}_{ϕ}}^{ϕ} , & {m}_{ϕ} - {n}_{ϕ} \\ \text{?} & \text{?}\end{matrix}$$

$$\text{?} \qquad ( C13 )$$

$$\text{?} \qquad ( C14 )$$

$$\text{?} \text{indicates text missing or illegible when filed}$$

and the Z_{nz}^{Crϕ,ϕz,rz} “coupling” matrices define the interactions between rings and are given by:

$${Z}_{{n}_{z}}^{Cr ϕ} ( {m}_{ϕ} , {n}_{ϕ} ) = j ω {M}^{r ϕ r ϕ} , \qquad ( C15 )$$

$${Z}_{{n}_{z}}^{C ϕ z} ( {m}_{ϕ} , {n}_{ϕ} ) = \{ \begin{matrix}- {Z}_{{n}_{z} {m}_{ϕ}}^{ϕ} + j ω {M}^{ϕ z ϕ z} , & {m}_{ϕ} = {n}_{ϕ} \\ 0 , & otherwise\end{matrix} \qquad ( C16 )$$

$${Z}_{{n}_{z}}^{Crz} ( {m}_{ϕ} , {n}_{ϕ} ) = \{ \begin{matrix}{Z}_{{n}_{z} ( {m}_{ϕ} + 1 )}^{r} , & {m}_{ϕ} = {n}_{ϕ} \\ 0 , & otherwise\end{matrix} \qquad ( C17 )$$

The additional mutual inductance term (M^{rϕrϕ}) describes the coupling due to magnetic flux between pairs of rϕ meshes on adjacent rings. The interactions between differently oriented mesh groups are:

$${Z}^{msh} = [ \begin{matrix}{Z}_{1}^{Rmsh ,} & & - {Z}_{2}^{Cmsh ,} & 0 & 0 \\ 0 & & ⋱ & & 0 \\ 0 & & & & - {Z}_{{N}_{z} - 1}^{Cmsh} \\ - {Z}_{{N}_{z}}^{Cmsh} & 0 & 0 & & {Z}_{{N}_{z}}^{Rmsh}\end{matrix} ] , \qquad ( C18 )$$

$$msh = ϕ zr ϕ , rzr ϕ , rz ϕ z$$

$$where the {N}_{ϕ} \times {N}_{ϕ} {Z}_{{N}_{z}}^{Rmsh} matrices are :$$

$${Z}_{{n}_{z}}^{R ϕ zr ϕ} ( {m}_{ϕ} , {n}_{ϕ} ) = {Z}_{{n}_{z}}^{C ϕ zr ϕ} ( {m}_{ϕ} , {n}_{ϕ} ) = \{ \begin{matrix}{Z}_{{n}_{z} {m}_{ϕ}}^{ϕ} + j ω {M}^{r ϕϕ z} , & {m}_{ϕ} = {n}_{ϕ} \\ 0 , & otherwise\end{matrix}$$

$${Z}_{{n}_{z}}^{Rr zr ϕ} ( {m}_{ϕ} , {n}_{ϕ} ) = {Z}_{{n}_{z}}^{Crzr ϕ} ( {m}_{ϕ} , {n}_{ϕ} ) = \{ \begin{matrix}\text{?} \\ \text{?}\end{matrix}$$

$$\text{?} \qquad ( C19 )$$

$$\text{?} \qquad ( C20 )$$

$$\text{?} \qquad ( C21 )$$

$$\text{?} \text{indicates text missing or illegible when filed}$$

The additional mutual inductance term M^{rϕϕz} and M^{ϕzϕz} describes the coupling due to magnetic flux interaction between pairs of rϕ and ϕz, and ϕz and ϕz, oriented meshes with shared current paths through the Z_{nzmϕ}^{ϕ} impedances, respectively. Periodic boundary conditions for the matrices are applied by making the condition that if m_{ϕ,z}=N_{ϕ,z}+1, then m_{99 ,z}=1 and if m_{ϕ,z}=n_{ϕ,z}−1, where n_{ϕ,z}=1, then m_{ϕ,z}=N_{ϕ,z}.

The parameters of the network model are closely related to the line inductances and capacitances for small segments of transmission lines. Thus, reasonable values could be determined from the corresponding analytical or empirical equations. Alternatively, as employed here, the variables are set to match the standard of full-wave eigenmode simulation of the dispersion relation in the stepwise method described in this section. The analogous electrostatic or transmission-line equivalent model will be referenced for comparison to the final value. The discrepancy in the values from the equivalent circuit model and those predicted by the transmission line or electrostatic models is to be expected due to the limited applicability of the equations for high frequencies and geometries and current or charge distributions that do not correspond exactly to those of transmission lines. Additionally, all equations used are approximations based on transmission lines in free space, but here a 1.56 mm thick substrate that is slightly wider than the ring conductor is placed on the opposite side of ground with εₛ=3.4 (See FIG. 13).

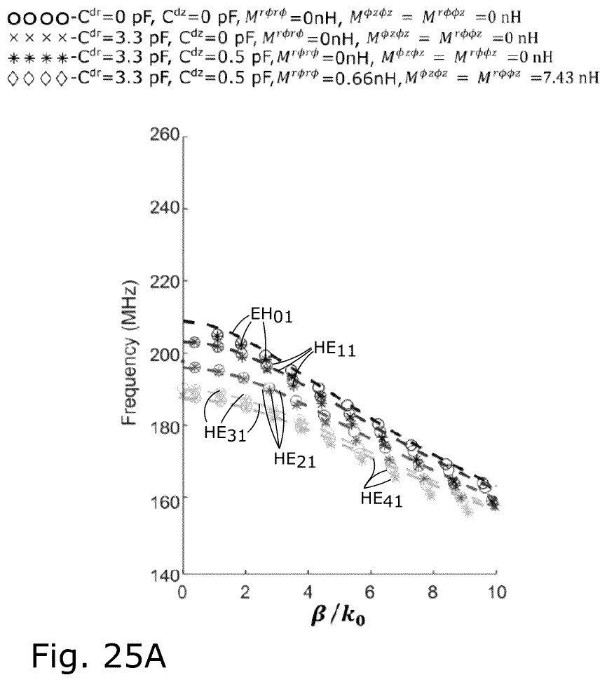

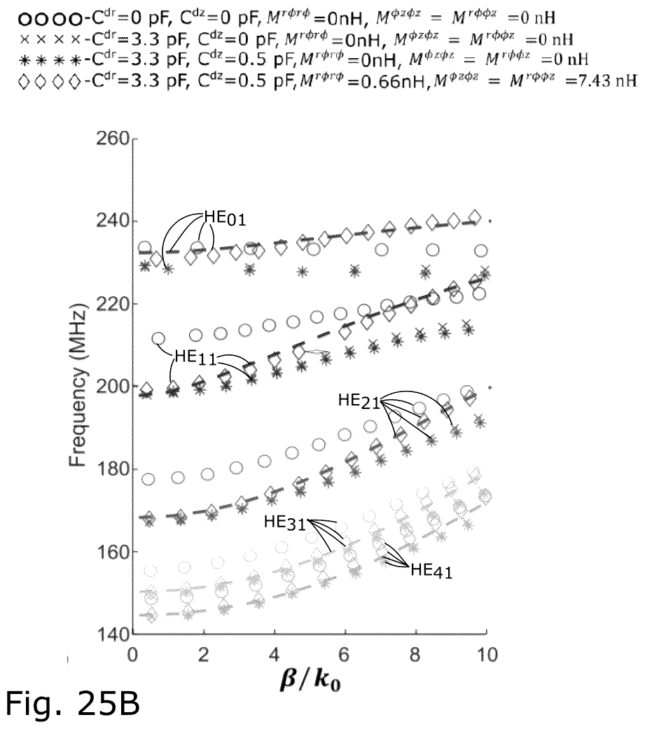

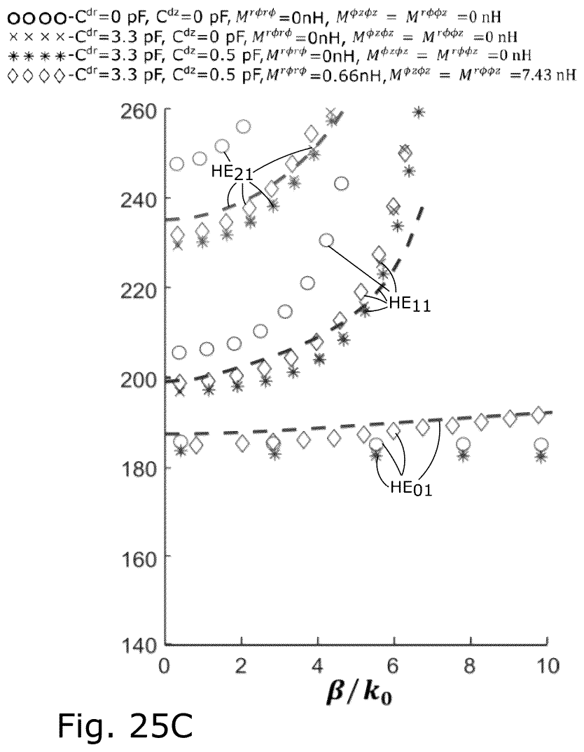

The number of rings (N_{z}), which is used in the evaluation of the dispersion, was set to 64 for this analysis. To tune the network model to match simulation the geometry-dependent parameters (C^{rd}, C^{zd}, M^{rϕrϕ}, M^{ϕzϕz}, M^{rϕϕz}) are all initially set to zero. Initial values L^{rG}=2.5 nH, C^{ϕd}=0.5 pF , L^{zG}=19 nH are set-in anticipation of later adjustments to match the separation of HEₙ₁ modes for the low-pass MNL case, the separation of the modes and the correct slope in the ENNZ case. The value for L^{zG} is obtained by quasistatic equations for a 50 mm long, 12.5 mm wide, 10 mm height above ground microstrip line is 19 nH. Additionally, resistances of 0.0145 Ω and 0.0210 Ω are placed in series with each L^{z} and L^{ϕ}, respectively, according to the skin depth of copper at 200 MHz (4.61 μm for a resistivity of 1.678 μΩcm), width of 12.5 mm and lengths of each segment (50 mm and 72 mm). With each step in the variation of geometric parameters described below, the impact on the dispersion and cut-off frequencies with relation to the simulated values can be observed by referencing FIG. 25.
Step 1: L^{ϕG} is set to match the cut-off frequencies of the MNL cases. For the two MNL cases the cut-off frequency of the HE01 mode (ƒf₀ₘₚ) is estimated by

$${f}_{0 mp} ∼ \frac{1}{2 π \sqrt{3 {L}^{ϕ} ( {C}^{ϕ} + {C}^{ϕ d} ) / 2}} , \qquad ( C22 )$$

due to three series L^{ϕ} inductors for each C^{ϕ} in the equivalent circuit. Setting L^{ϕ}=31nH results in ƒ₀ₚ=228 MHz and ƒ₀ₚ=183 MHz for the HP-MNL and low-pass MNL cases, respectively. The L^{ϕ} is in the middle of the range predicted by quasistatic and empirically derived equations for a 72-mm-long, 12.5-mm-wide, 10 mm-height above ground microstrip line (27.4<L^{ϕ}<34.9 nH).
Step 2: For the ENNZ case, where jωL^{ϕ}<<1/jω(C^{ϕ}+C^{ϕd}), the cut-off frequency of the EH₀₁ mode (ƒp) is

$${f}_{p} ∼ \frac{1}{2 π \sqrt{{L}^{r} ( {C}^{rd} + 2 {C}^{rd} ( {C}^{ϕ} + {C}^{ϕ d} ) / ( {C}^{p} + {C}^{ϕ} + {C}^{ϕ d} )}} \qquad ( C23 )$$

The C^{rd}=3.3 pF is set to closely match the simulated value of ƒₚ=207 MHz. The capacitance predicted by quasistatic equations for a microstrip with that geometry is 2.1 pF.
Step 3: To match the slope of the dispersion curve for the ENNZ case a value of C^{zd}=0.5 pF is used. The predicted capacitance between two 12.5 mm wide coplanar strips 72 mm in length separated by 50 mm is C^{zd}=0.67 pF.
Step 4: The mutual impedance terms are modified so that the slope of the HE₀₁ dispersion curve matches that in simulation for both the HP-MNL and LP-MNL cases. M^{rϕrϕ}has a low value (M^{rϕrϕ}=−0.01) due to the large separation between rings relative to liner thickness and therefore has only a small effect in increasing the cut-off frequencies, but in more closely spaced rings or thicker liners it would be a critical parameter significantly altering the dispersion slope and cut-off frequencies. For the MTM liner discussed herein, the inclusion of the mutual impedance terms M^{ϕzϕz}=M^{rϕϕz}=−0.225 is a factor for obtaining the correct positive dispersion slope of the HE₀₁ mode, which would otherwise be negative.

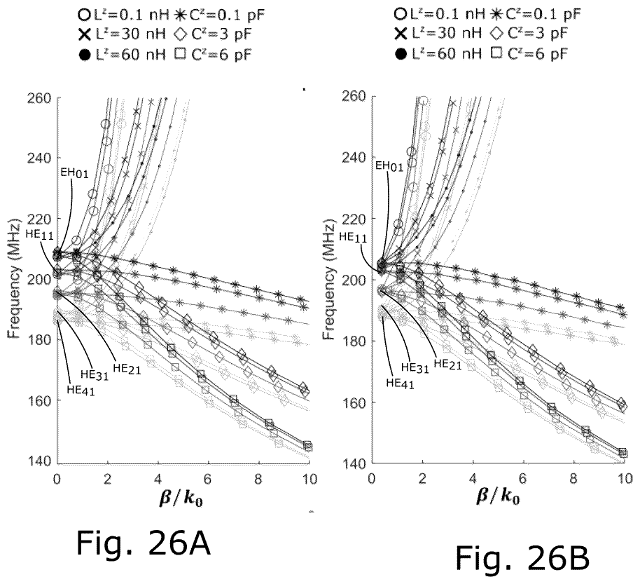

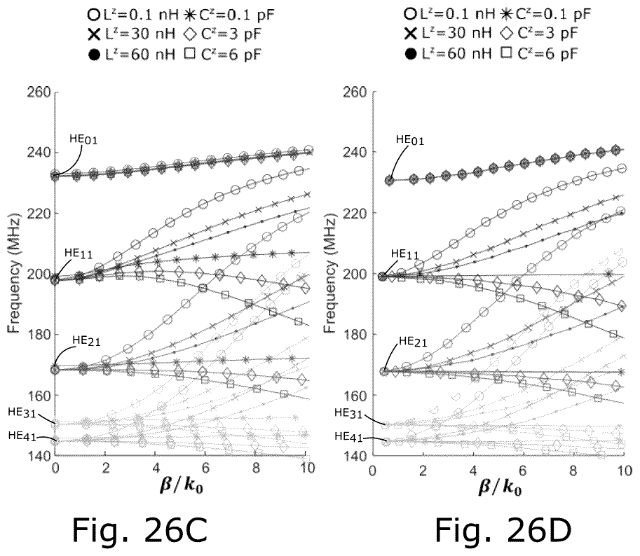

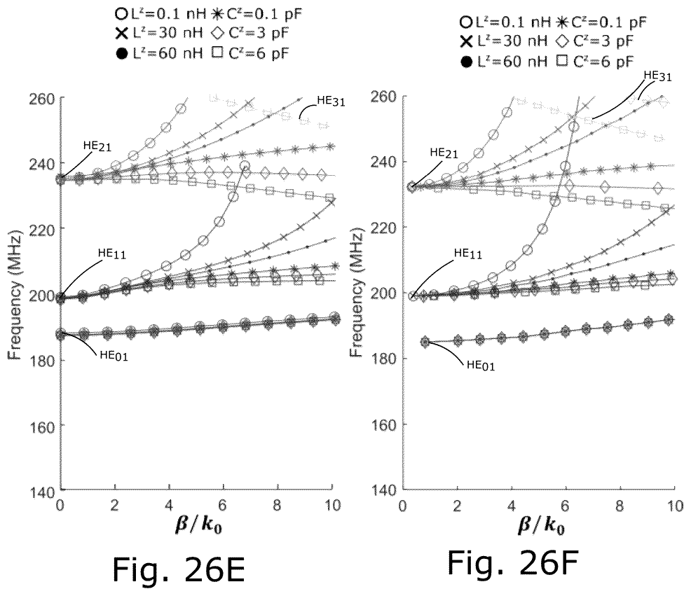

Earlier herein it was demonstrated that flat dispersion curves near cut-off result in mixing of the first few HE₁₁ longitudinal resonances for a practically sized liner. The variation of the longitudinal tuning impedances is performed here to determine which values result in larges slope in the dispersion for the three cases, while avoiding mixing of different azimuthal modes. The dispersion curves calculated by the network model or by simulation with the HFSS eigenmode with are shown in FIG. 26. The cut-off frequencies and relative change in the slope with varying longitudinal impedances agree closely between the two methods, further validating the accuracy of the network model for representing the MTM liner.

The ENNZ case has the greatest slope of the HE₁₁ dispersion curve when C^{z}=6 pF, and there is no overlap of other HEₙ₁ modes. However, the EH₀₁ mode does overlap. Since these modes are orthogonal it may be possible to excite the HE₁₁ mode largely independently of the EH₀₁ mode, but the ENNZ case also requires the use of large highly lossy inductors (L^{r}), which reduces the achievable transmit efficiency. The spacing of the HEₙ₁ modes is also much less than the other cases (dependent on C^{ϕ}).

Although varying the longitudinal impedance allows for greater slope of the dispersion curve at cut-off, in cases where overlap of HEₙ₁ modes propagating at the same frequency means that multiple modes will nevertheless be excited simultaneously. This is the case for the MNL low-pass case in which the largest slope occurs with L^{z}=0, but the HE₁₁, HE₂₁ and HE₃₁ modes all propagate at the 4.7 T Larmor frequency of 200 MHz. Thus, this case should be avoided and the L^{z}=30 nH case is preferred. However, for practical implementation this requires the use of lossy inductors (L^{z} as well as L^{r}). For the HP-MNL the L^{z}=0 case results in the largest slope, but since the HE₀₁ mode is unaffected by the longitudinal tuning impedances there is no overlap of HEₙ₁ modes at 200 MHz, making this the most desirable case for practical implementation.

In the claims, the word “comprising” is used in its inclusive sense and does not exclude other elements being present. The indefinite articles “a” and “an” before a claim feature do not exclude more than one of the feature being present. Each one of the individual features described here may be used in one or more embodiments and is not, by virtue only of being described here, to be construed as essential to all embodiments as defined by the claims.

1. A liner for a bore of an MRI scanner having a magnetic field, the liner comprising:
plural conductors extending circumferentially within a liner region of the bore;
circumferential impedances on the plural conductors; and
radial impedances connecting the plural conductors to a radially outer conductive structure.

2. The liner of claim 1 in which the radially outer conductive structure comprises outer conductors extending circumferentially within the bore and outer circumferential impedances on the outer conductors.

3. The liner of claim 2 in which the outer conductors are connected axially by outer axial electrical connections.

4. The liner of claim 3 in which the outer axial electrical connections include impedances.

5. The liner of claim 1 in which the radially outer conductive structure comprises an outer conductive shield of the bore.

6. The liner of claim 1 in which the plural conductors are connected axially by inner axial electrical connections.

7. The liner of claim 6 in which the inner axial electrical connections include impedances.

8. The liner of claim 1 in which the circumferential and radial impedances are selected to produce an effective negative and near zero permittivity within the liner region of the bore.

9. The liner of claim 1 in which the circumferential and radial impedances are selected to produce a large negative permeability and effective positive and near zero permittivity within the liner region of the bore.

10. The liner of claim 1 in which the liner extends less than a full length of the bore.

11. The liner of claim 1 further comprising a cable connected to the liner to generate an MRI excitation field within the liner.

12. The liner of claim 11 further comprising one or more additional cables connected to one or more respective additional points within the liner.

13. The liner of claim 12 in which the one or more cables are connected to RF power supplies with different phase and/or power.

14. The liner of claim 1 in combination with an antenna within the bore to generate an MRI excitation field within the liner.

15. The liner of claim 1 in combination with plural antennas within the bore to generate an MRI excitation field within the liner, the plural antennas being connected to RF power supplies with different phase and/or power.

16. The liner of claim 1 in which the plural conductors are formed in different sets, conductors of the different sets alternating within the bore, and the different sets respectively arranged to produce a desired propagating mode at respective different MR frequencies.

17. The liner of claim 1 in which the plural conductors are formed in different sets, conductors of the different sets alternating within the bore, and the different sets respectively arranged to produce different propagating modes.

18. The liner of claim 17 arranged to produce the different propagating modes at a single frequency.

19. The liner of claim 18 in which the different propagating modes are separately excitable with common pulse shapes for shimming of the RF magnetic field within the bore at the single frequency.

20. The liner of claim 18 in which the different propagating modes are separately excitable with different pulse shapes.

21. The liner of claim 1 in which the magnetic field has a nominal strength of 1.5 T, 3 T, 4.7 T, 5T or 7 T.
